(19) United States
(12) Patent Application Publication
(10) Pub. No.: US 2004/0014097 A1
(43) Pub. Date: Jan. 22, 2004

(54) Genetic test apparatus and method

(76) Inventors: Ronald C. McGlennen, Edina, MN (US); Naomi M. Williamson, Fridley, MN (US); Aaron Franks, Hopkins, MN (US); Sabine Holmes, Minneapolis, MN (US); Robert P. Schuldt, Eagan, MN (US)

Correspondence Address: GRAY, PLANT, MOOTY, MOOTY & BENNETT, P.A., P.O. BOX 2906, MINNEAPOLIS, MN, 55402-0906, US

(21) Appl. No.: 10/409,337

(22) Filed: Apr. 7, 2003

Related U.S. Application Data
(60) Provisional application No. 60/378,418, filed on May 6, 2002.

(30) Foreign Application Priority Data
Sep. 18, 2002 (WO) PCT/US02/29628

(51) Int. Cl. G06F 17/60; C12Q 1/68; G06F 19/00; G01N 33/48; G01N 33/50

(52) U.S. Cl. 435/6; 702/20; 705/3

(57) ABSTRACT

The invention involves creation of an integrated genetic testing kit. The kit combines materials and components to aid in performance of each of the steps in a genetic assay. Additionally, the invention describes a variety of electronic tools and adjunct materials to make easier the collection and organization of patient related information that is used in interpretation of the analytic genetic data. One aspect of this invention is as a stand-alone integrated test kit, the test results of which may be in the form of fluoroscopic output. Another aspect of this invention is a telemedicine model. In this model, the invention is a larger genetic testing system in which procurement of genetic material, its testing, and its interpretation may occur at different locations. The telemedicine model comprises the integrated kit, instrumentation, and an electronic transmission system for delivery of the genetic test data to a remote data system where it is interpreted and a report is generated.

### CROSS-REFERENCE TO A RELATED PATENT APPLICATION

This application claims the benefit of the filing date of U.S. Provisional Patent Application Serial No. 60/378,418, filed May 6, 2002 and of International Patent Application Serial Number PCT/US02/29628, filed Sep. 18, 2002.

### FIELD OF THE INVENTION

This invention concerns systems, apparatus, and methods to simplify and improve genetic testing, analysis, and consultation. The invention includes the capability to permit the sampling and testing of genetic source material at a location different from the analysis of the results and the preparation of consultative reports based on the results.

This invention also describes the design, assembly, and logistics of an integrated test kit to be used in gene based testing. The integrated test kit aids laboratories to perform the technical aspects of a genetic test by providing a convenient, easy to use, test process and apparatus, the use of which results in improved quality of the genetic test results. The kit may be used alone or in conjunction with a genetic testing system that includes Internet based analysis of the results and preparation of consultative reports based on the results.

### BACKGROUND

Generally, most genetic tests involve five primary steps or processes: 1) specimen procurement; 2) nucleic acid purification; 3) enzymatic manipulation of one or more gene loci by means of some type of genetic chemistry; 4) analysis of the derived data output of the gene chemistry; and 5) interpretation and reporting of the results. While there are many individual genetic tests, there are at least four categories of medical applications of genetic testing, including tests for (1) constitutional or inherited disorders; (2) acquired genetic disease, such as cancer; (3) molecular characterization of infectious organism; and (4) disease of genetic predisposition. The level of technical skill to perform these tests as well as the medical expertise to interpret this information into usable results are different.

Although there has been a large increase in the number of genetic tests available, the majority of existing tests are for rare and highly esoteric types of diseases. The complexity and the historically high costs of each test have caused the majority of genetic testing to be performed in only a relatively small number of centralized or reference laboratories. Other than specimen procurement, which is typically performed at the point of care such as a local laboratory, hospital or physician office, these centralized laboratories perform all of the remaining processes for a particular genetic test within their laboratory location. However, through the integration of the respective materials and reagents as well as with improvements in the testing procedure into an integrated kit, this invention makes possible the performance of these specialized laboratory tests in a majority of clinical laboratory settings. The invention, an integrated testing kit, simplifies the processes of specimen procurement, nucleic acid purification, genetic chemistry and other steps leading up to the interpretation of the test to a point where no specialized training or unique laboratory skills are required. With the capability to perform these tests at more laboratories, and hence at places closer to the point of care the invention will improve both the availability of these healthcare services and their cost effectiveness. The aim of this invention is to make available state-of-the-art genetic tests to smaller, less sophisticated and nonspecialized laboratories close to a larger number of patients who require such tests. Whereas this invention has immediate application in such settings as smaller hospitals and physicians' offices, eventually it may be useful for consumer-driven places such as pharmacies and specialized healthcare kiosks.

Molecular diagnostics involves the characterization of human disease by examining nucleic acids, both DNA and RNA, which are the template for all proteins that mediate disease. Currently, molecular diagnostics involves the use of a variety of technical approaches to extract, modify, and analyze DNA or RNA for changes inherent to the nucleotide sequence that make up the genome. These changes, called mutations or polymorphisms, are the basis for determination of who we are as humans and the differences between us, some of which give rise to disease.

### SUMMARY OF THE INVENTION

To bring sophisticated genetic testing to locations previously thought to be unsuited to support such services, the invention divides the various steps in genetic tests into those performed at sites near to where the sample is collected, and those related to where the test is interpreted. A telemedicine model is employed, so that the test results are transmitted to a central location where the testing expertise resides. An integrated kit of this invention is used to perform the various genetic tests at a site near where the sample is collected.

Specifically, the invention involves systems and methods to simplify and improve the genetic testing process and to permit the secure and effective testing of genetic source material at different locations from the analysis of the results and the preparation of interpretive genetic testing reports thereby permitting such tests to be performed by more clinical laboratories. The invention also involves the gathering of additional information on the patient as well as systems and methods to use such information in conjunction with the genetic testing results to provide more thorough and useful physician and patient results and feedback.

The invention may be embodied as a controlled system of computerized hardware, software, communications links, genetic and medical expertise and quality control to ensure test and report accuracy, quality and patient privacy. This could include systems and methods which employ networked, computerized equipment for all or part of the processes of: specimen procurement and nucleic acid purification; genetic chemistry; data collection; data verification; data transmission; interpretation of data; and reporting results of the interpretation. The particular selection of network hardware or software is not critical to the scope of the invention, except as specifically described below.

Another aspect of the invention is a genetic test data gathering process and system that can, either automatically or initiated by a remote site or central location, gather from a remote site database all relevant genetic data generated through genetic chemistry, such as both PCR genetic data and/or non-PCR genetic data.

Another aspect of the invention is a genetic test data gathering process or system that, based on the genetic test requested and other relevant factors, determines patient and other relevant information to gather or request regarding the patient. Such data could include, but not be limited to: patient billing information; other patient genetic data; standard medical record data; characterizations of the patient's physical state of health, past laboratory tests, physical exam or specialized studies; commentary from qualified medical professionals and other information relevant to the patient's family history and genealogy; information regarding the patient's environmental context (e.g., where they live, environmental effectors such as hazardous material exposures and climatic factors); and any other information that, when correlated with the genotype data, improve the predictive value of genomic testing as compared to consideration of such data in isolation. In a preferred embodiment, the process or system can (automatically or as initiated by the remote site or central location) gather or parse from any remote site database all relevant genetic data from whatever other genetic technology data that are available (e.g., photographic or digital images data from colorimetric; fluorometric, radioisotopic or other evoked biomolecular signal outputs). In another preferred embodiment, the process or system can (either automatically or as initiated by the remote site or central location) automatically gather or parse relevant patient data from the remote site databases to the extent such information is available. In another preferred embodiment, the process or system makes requests from the test requester (e.g., physician) and/or the relevant patient data, in the form of system generated written consent forms, a form filled out on the Internet, or an email to the testing facility, or any other equivalent data gathering technique. In another preferred embodiment, the process or system may gather the raw or unprocessed relevant patient data when other necessary elements of data are ready to be transmitted.

Another aspect of the invention is a genetic test data verification sub process or subsystem that determines the suitability of the genetic testing information gathered by the genetic test data gathering process or system. For example, this may involve determination if other relevant patient data gathered by the genetic test data gathering process or system is adequate, complete and otherwise ready for transmission. In another example, the system or process may generate a report at a remote site location that explains any data errors or inconsistencies and recommendations for corrections, if the relevant patient data gathered by the genetic test data gathering process or system is not adequate, complete and otherwise ready for transmission.

Another aspect of the invention is a data transmission preparation sub process or subsystem that prepares for electronic transmission of some or all relevant patient data that has been verified by the genetic test data verification process or system. In one preferred embodiment, prior to transmission, all information identifying the patient is masked and/or encrypted to prevent patient identification. In another preferred embodiment, prior to transmission, the subsystem or sub process separates all data gathered into a plurality of two files. For example, one file could contain non-confidential information identifying the patient and another file could contain other confidential information but not patient identifying information. One or both of these files could be encrypted by the system. In another preferred embodiment, prior to transmission, all data gathered is simply encrypted without substantial further modification.

Another aspect of the invention is a data transmission sub process or subsystem that transmits all data gathered to a central location. The data transmission may be initiated by the remote location or by the central location. In either case, separate files generated and/or encrypted as described above may be transmitted at different times and/or in different communication channels (e.g., one or more virtual private network(s) could be employed for separate files). It is preferred but not required to assemble data into batches for fast and efficient transmission to the central location.

Another aspect of the invention is, at a central location, an interpretation process or system that receives patient information electronically and performs an initial validation of the information. For example, a preliminary genetic testing diagnosis may be performed for review and validation by a qualified health professional. It is preferred that, following preliminary genetic testing diagnosis, the interpretation process or system will then include automatic review and analysis of the genetic test results (in combination with all other relevant patient information), including review and validation by a qualified health professional.

In another preferred embodiment, an expert system generates natural language explanations regarding particular genetic tests. These explanations may be used for various purposes, including (without limitation): imparting the appropriate clinical application and significance of such tests; providing answers to specific queries about the use of such tests; and providing formal, context-specific interpretation of results of such tests when they have been applied to individuals.

A preferred (but not required) embodiment of the expert system comprises one or more components. One such component is an expert database containing up-to-date knowledge about relevant genetic conditions. The subjects of such knowledge could be abnormalities arising from the human body's expression of certain genetic patterns; the underlying mechanism that causes such expression; the impact of human states such as genotype, gender and age on the likelihood and degree of expression of these abnormalities; the impact of medications, treatments, diets, and life choices on the likelihood and degree of expression of these abnormalities; recommended adjustments to standard care practices deemed or believed advisable due to such expression; recommended general health practices for those with the potential for expressing such abnormalities; recommendations for further testing to more completely characterize any genetic explanation for an abnormality; and health-related recommendations for relatives of individuals with known genotypes. Another component is an interface to an electronic system that has access to requests for explanations about genetic tests, abnormalities expressed by genetic abnormalities, and the effect of life states, practices, and choices on the likely expression of these abnormalities. A further component is an interface to an electronic system that contains demographic information and specific state information regarding individuals being tested for genetic abnormalities and their resulting genotype determined by this testing. Furthermore, the expert system may comprise a data storage subsystem that temporarily holds information that has been passed to the interfaces described above, and makes it available to the control mechanism described below. Another component of the expert system may be a control mechanism that inspects the contents of the data storage subsystem and, based on the contents, assembles appropriate data from the expert database into a coherent explanation or interpretation. Yet another component is a display and/or reporting output module for rendering the output of the database so that is viewable or made part of a printable report.

In one preferred embodiment, the expert database is divided into logical compartments that correspond to relevant elements of a genetic testing ontology for each particular genetic test. In another preferred embodiment, the information stored in one or more logical compartments includes variable components that can be rendered or not rendered as output, depending on state information pertaining to the subject being tested, and under the control of the control mechanism described above. In another preferred embodiment, the expert database allows a content expert (such as a genetic counselor) to add information to one or more of the compartments and thus make such information available for inclusion in explanations and interpretations, without the need of additional intervention by computer programming personnel.

Another aspect of the invention is a reporting sub process or subsystem that, following the interpretation of testing, automatically generates a report of the interpretive genetic test results in medical terms. It is preferred but not required to additionally include a comprehensive report containing the interpretive genetic test results that states in medical and/or genetic terms the result of the analytic test, and further comprises a comment section that may contain at least some of: a statement that recognizes the patients contextual information and what impact if any the genotype for the disease being tested has on this contextual data; a statement concerning disease risk, the modification of that risk given the genotype result, and the contextual data (given that such a risk modification is known); and a statement of the implications that may exist for therapy or prognosis. A simplified process of specimen procurement, nucleic acid purification, genetic chemistry and other steps leading up to the interpretation of the test to a point where no specialized training or unique laboratory skills are required, collectively provide the to perform these tests at a much larger number of laboratories, and hence at places closer to the point of care. This will improve both the availability of these healthcare services and their cost effectiveness.

In a preferred embodiment, information (including the reports described above) may be entered into a database that may be accessed only at the remote testing site, using any electronic or other technique (e.g., password-type authorization, over the Internet, using direct dial-up, and so on). In another preferred embodiment, the central location transmits the reports to the remote location (or the patient) either by hard copy or electronic document transmission techniques (e.g., email with or without document attachments).

The high quality of the data generated from the invention is intended to enhance the interpretation of the genetic test data into a series of reports. One embodiment of the invention is where it is used as part of a more comprehensive genetic testing system, where the invention facilitates the extraction of the sample-derived nucleic acid and the gene chemistry steps as well as the presentation of the results through a series of analytic instruments such as, but not limited to a fluorometer interfaced with a computer and connected to the Internet. The invention can be used with a fluorometer or similar analytic instrument that generates data locally, i.e. on site or as part of an integrated genetic testing system that includes transmission of various types of data to a remote computer and database, where a qualified healthcare professional provides the expert interpretation and composition of the various reports. Through that same system, namely a secured Internet portal, the invention is used to monitor quality control of the test results being produced, to track inventory of the kit elements, and access to essential information such as material safety data, storage and outdating of the reagents.

The integrated genetic test kit of this invention is a specially designed genetic testing kit (including an improvement of existing kits) that can accommodate a variety of specimen types, manners of procuring those specimens, and includes components for both the nucleic acid purification and the gene chemistry required to achieve a genetic test result. The integrated genetic test kit is organized in a manner that simplifies the technical and operational aspects of the steps required to perform most molecular genetic assays. The kit may be used by as a stand-alone device or as part of the integrated genetic testing system heretofore described. In a preferred embodiment, nucleic acid purification is enhanced to increase the likelihood of a high quantity of input nucleic acid from only a minimal sample of matter containing DNA or RNA. In another preferred embodiment, the instructions for purification of nucleic acid by its purification from a sample or specimen are enhanced to perform the assay based on the use of a rapid nuclei extraction method. In yet another preferred embodiment, the kit simplifies the (PCR or non-PCR) genetic chemistry steps of the genetic testing protocol through the use of better assay controls and simplification of the operation of the protocol. One manner of accomplishing this improved assay protocol is through the selection of equipment and configuration of the assay (e.g., arrangement of extractor and mixing racks and other kit elements) to make following the details of the procedure easier. In another preferred embodiment, the kit improves upon the non-PCR genetic chemistry technology by implementing one or more of the following: (i) configuring the assembly of the various reagent mixes; (ii) prealiquoting control samples into respective microtitor wells for the user; and (iii) improving control samples by basing control samples on the use of genomic DNA or RNA.

The integrated genetic kit of this invention comprises a set of specialized components, such as the necessary reagents and disposable materials to perform a DNA or RNA based test. These tests include any of a series of localized gene sequence alterations including detection of nucleotide substitutions through mutation, single nucleotide polymorphisms, and small nucleotide deletions or insertions. The kit includes a series of devices to house, and make easier to use, the various reagents for each of the test procedures including: devices for the collection of specialized specimens, materials for sample labeling, forms for test requisition, and specialized “racks” for guiding the procedure for extracting purified nucleic acid from a sample and gene chemistry, and work flow management. The kit also includes a detailed procedure manual that combines what have been separate process steps, protocols, and procedures into an integrated documented set of process steps. The procedure manual of this invention employs several unique strategies to communicate an otherwise complex and sometimes apparently overlapping set of process steps into straightforward, step-by-step recipes or formulas. The procedure manual also includes instructions for accessing and using several optional component tools of this invention, available on a password accessible web-site, that simplify the process of making calculations for reagent volumes and estimating time relevant to aspects of the procedure. The procedure manual includes forms for gathering patient specific information that enhances the value of the interpretative genetic test report. The invention further includes the manner of labeling elemental components in the kit for the purpose of quality control and tracking of reagent lot numbers, usage, and expiration dates. The labeling, along with the design of the respective racks and materials, make possible the restocking of the kit through an optional electronically registered supply chain monitoring system. The integration of the physical kit with the labeling of the elemental components and the linkage of said elements to an optional electronic supply and information management monitoring system is all intended to make for higher quality test data and the subsequent interpretative medical report of the test results for patient care.

### BRIEF DESCRIPTION OF THE DRAWINGS

The accompanying drawings show a particular embodiment of the invention as an example.

They are not intended to limit the scope of the invention. For example, while the invention is shown and described in schematic terms, computer hardware, or software in any combination may perform many aspects of the invention.

FIG. 1 is a schematic diagram of one telemedicine embodiment of the invention.

FIG. 2 is a schematic diagram of an embodiment of an operational scheme for genetic testing, in accordance with the invention.

FIG. 3 is a schematic diagram of an embodiment of the integrated genetic test kit.

FIG. 4 is a schematic diagram of an embodiment of an extractor rack for nucleic acid purification, setup for use of the Gentra Generation extraction system and reagents.

FIG. 5 is a schematic diagram of an embodiment of the extractor rack of FIG. 4, with specimens, reagents, and solution containers in apertures or slots in the extractor rack.

FIG. 6 is a schematic diagram of an embodiment of a mixing rack, setup for use of the Invader assay system and reagents.

FIG. 7A is a schematic diagram of an embodiment of a Factor V Leiden microtitor template guide for the addition of reagents into a 96 well microtiter plate, setup for use of the Invader reaction system and reagents.

FIG. 7B is a schematic diagram of an embodiment of a Factor II microtitor template guide for the addition of reagents into a 96 well microtiter plate, setup for use of the Invader reaction system and reagents.

FIG. 8 is a schematic diagram of an embodiment of a microtitor template guide for mutant mix samples.

FIG. 9 is a schematic diagram of an embodiment of a form for ordering the Factor V Leiden and the Prothrombin 20210 mutation genetic tests for evaluation of inherited thrombophilia, which may be transmitted electronically to a remote site for fulfillment.

FIG. 10 is a schematic diagram of an embodiment of a form for entry of data into a tool that calculates reagent volumes and reaction times for the process of the invention using the Invader reagents, which may be transmitted electronically to and from a remote site for calculation.

FIG. 11 is a schematic diagram of an embodiment of an Internet based consultation and reporting aspect of the invention.

FIG. 12 is a schematic diagram of an embodiment of a form for entry of demographic and patient data used in the interpretation and reporting of the genetic test data, which may be transmitted electronically to a remote site.

FIG. 13 is a schematic diagram of an embodiment of the organization of an expert system for generating a report interpreting the genetic testing data based upon inputs from (i) the relevant medical literature identified by the expert system search function and its embedded search parameters, (ii) demographic patient information, and (iii) the expertise of a physician, which interpretive report addresses the particular set of disease associations, the corresponding and modifiable risk association, and the resulting therapeutic options.

FIG. 14 is a schematic diagram of an embodiment of a form of a customized interpretative report of the invention, which may be distributed electronically to the patient's physician.

FIG. 15 is a schematic diagram of an embodiment of a form of the interpreting physician's work list, which may be transmitted electronically.

FIG. 16 is a schematic diagram of an embodiment of a form of a summary batch analysis presenting an overview of customers, tests, and transactions, which may be transmitted electronically.

FIG. 17 is a schematic diagram of an embodiment of a page of the test protocol of the invention illustrating an embodiment of a reaction plate of the invention. The protocol may be organized such that there is an illustrative figure for each operational step.

FIG. 18 is a schematic diagram of an embodiment of a page of the test protocol of the invention illustrating the transfer of purified samples of patient nucleic acid to the wells of the reaction plate of the invention for a gene chemistry step of the protocol.

FIG. 19 is a schematic diagram of an embodiment of a page of the test protocol of the invention illustrating the transfer of control samples to the wells of the reaction plate of the invention, which wells contain patient sample nucleic acid.

FIG. 20 is a schematic diagram of an embodiment of a page of the test protocol of the invention illustrating the sequenced transfer of reagents in the mixing rack to create the master mix for the Factor V, Leiden and the Factor 11 genetic tests.

FIGS. 21A and 21B, together are a schematic diagram of an embodiment of two side-by-side pages of certain protocol steps of the invention illustrating (i) the manner in which certain protocol steps are displayed with text juxtaposed opposite a visual image a certain steps, (ii) the guide bar at the top of the embodiment of the protocol with a magnifying glass indicating the protocol steps addressed in FIG. 21A, and (iii) a patient sample tube and a mircrocentrifuge tube, both kit components, in the visual image of FIG. 21B.

### DETAILED DESCRIPTION

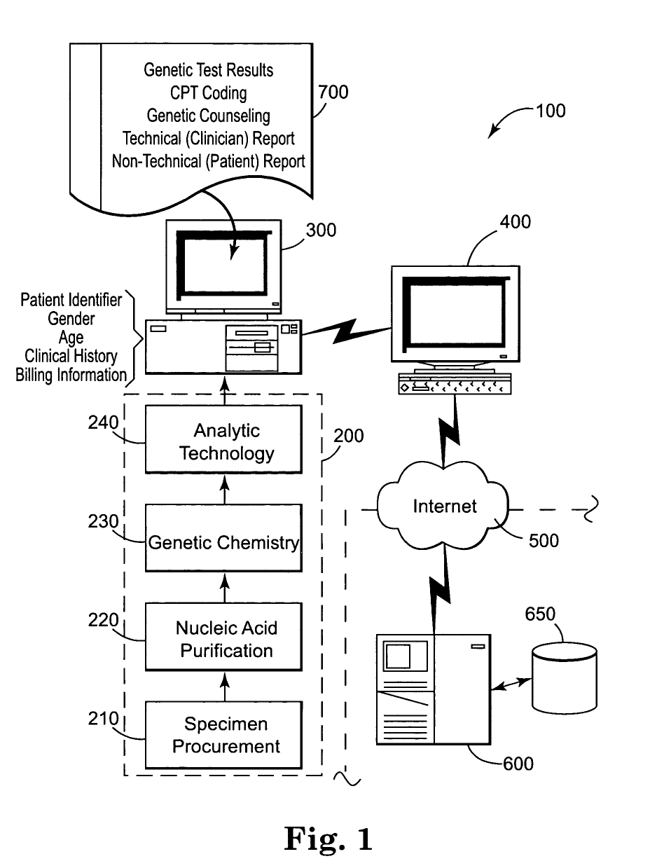

FIG. 1 is a schematic view of one preferred embodiment of the invention. In general functional terms, the system comprises eight major components: specimen procurement; nucleic acid purification; genetic chemistry; data collection; raw data detection and verification; transmission; interpretation; and reporting. The system may comprise discrete subsystems, each dedicated to a single functional component, or a fully integrated system. Similarly, any or all of the individual components may be integrated into sub-systems. Thus, the following description should not necessarily be understood to define any physical or functional separation of the components, except as specifically described and required.

In general terms, FIG. 1 shows a remote genetic testing system 100, comprising a clinical laboratory-based genetic testing system 200, a data collection system 300, a data transmission system 400, a computer network (shown by way of example only as the Internet) 500, a central data analysis/interpretation system 600, an expert database 650 and report data 700. Shown schematically as lightning bolts are conventional networking hardware and software as required to connect the various components of remote genetic testing system 100 together, according to known techniques not relevant to the scope of the invention.

The remote clinical laboratory-based genetic testing system 200 schematically comprises several subsystems, specifically specimen procurement subsystem 210, nucleic acid purification subsystem 220, genetic chemistry subsystem 230, and analytic technology subsystem 240. These are described in more detail below.

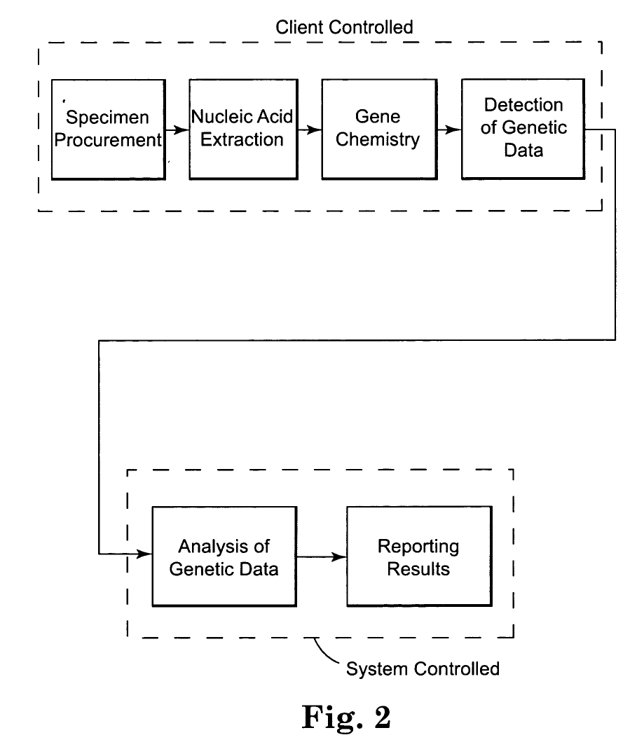

FIG. 2 schematically shows the genetic testing operational scheme of the invention and how the invention provides a local laboratory with the capabilities to perform genetic tests. In general terms, this involves the processes of nucleic acid extraction, gene chemistry detection of genetic data, interpretation of the genetic test data, and report generation. These steps are illustrated as separate and distinct because the invention improves upon this situation in that the collective steps can be reduced to a kit.

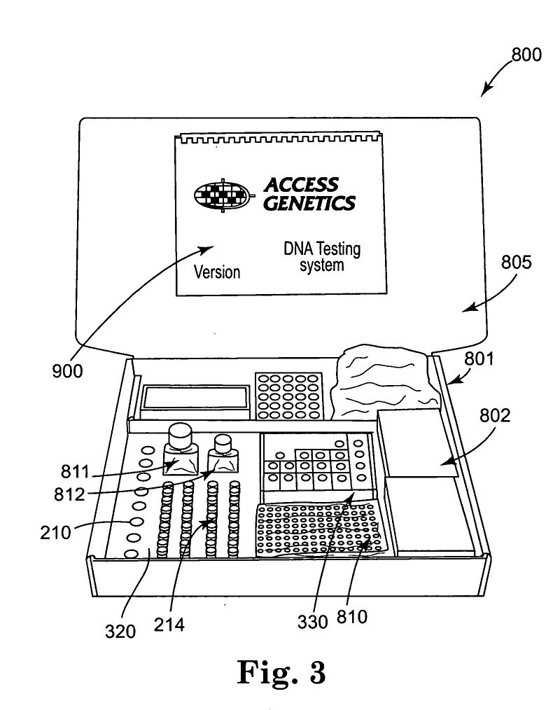

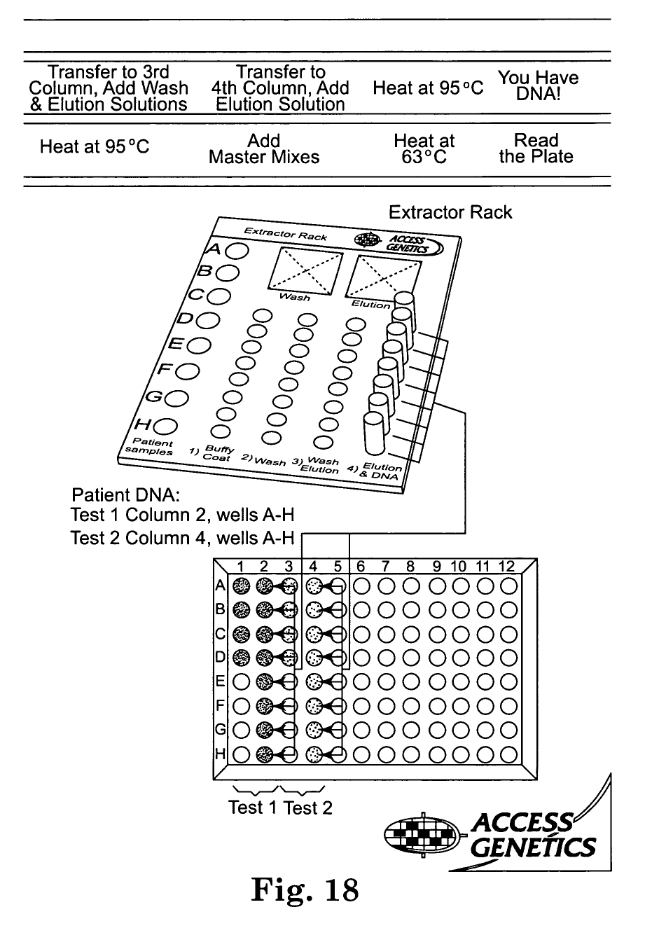

FIG. 3 illustrates one embodiment of the genetic test kit. In general terms, the kit includes the reagents 814 and materials necessary to execute the technical aspects of a genetic test from a single unified protocol of steps 330. As illustrated in FIGS. 3-8, one version of the genetic test kit includes the extractor rack 320 and reagent elements for DNA or RNA extraction, the mixing rack, reaction plate 810 and the components of the gene chemistry Invader in a newly organized presentation to make simpler the process of assembling the reaction components, the reaction plate and template guides and the corresponding replacement disposable plastic supplies. The last component of the test kit is the protocol 900 illustrated in FIGS. 17-21B that describes each of the steps in the technical aspects of the test, as well as for the initiation and completion of the data transmission steps of the system.

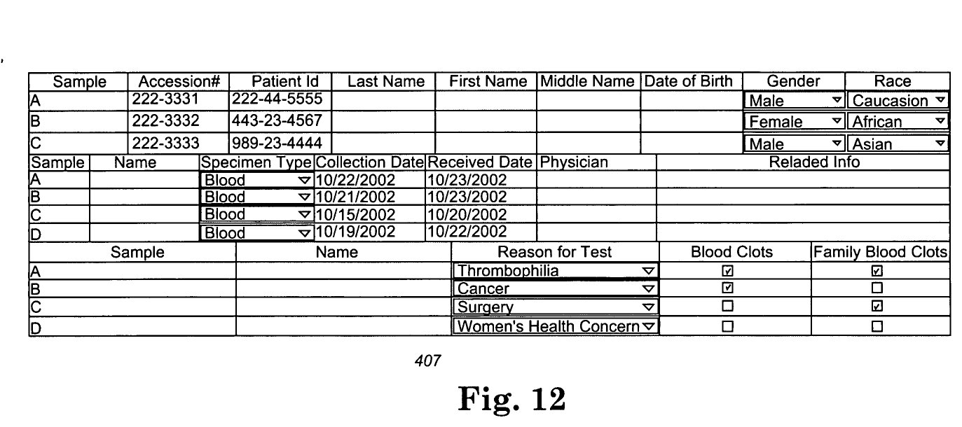

FIG. 11 is a schematic of the overall architecture of the telemedicine process of the invention. The process of data collection from an analytic instrument for detection of the genetic data resulting from the gene chemistry portion of the protocol occurs first at the computer of the remote site 400. Data is transmitted through the Internet via a process that is both secure and involves data that is encoded or dispersed in such as way as to render the patient information de-identified, 410. The transmitted analytic and patient specific demographic data is encrypted (e.g., 128-bit encryption or as otherwise desired) and unencrypted at the system firewall 420 and is collected at the central computer 430 which in turn provides the prompt to the persons interpreting the transmitted results to work on those data files. A qualified health care professional 440 interprets the data with the aid of the expert database 450. The completed test result is transmitted back to the site where the test was performed and printed or in some way distributed electronically to the requesters of the tests 460.

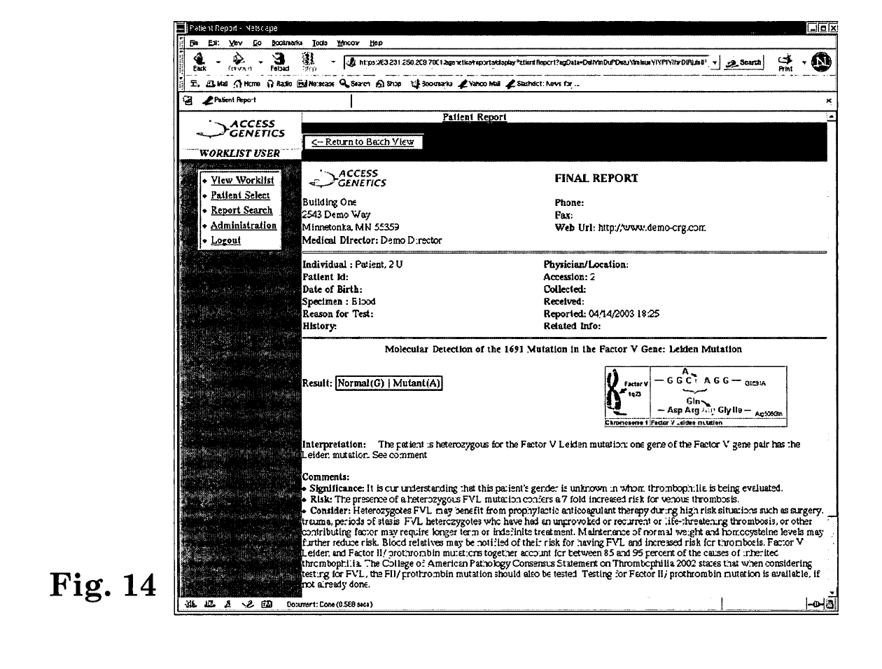

The expert system database 650 illustrated in FIGS. 1 and 13 used in the interpretation of the genetic tests consists of electronic platform such as a software data processing program with large amounts of abstracted medical information pertaining to aspects of genetic test interpretation. This may include subjects relating to disease assessment based upon the genetic data, medical conditions, risk assessment, other contributing gene and therapeutic options for the medical condition. The initiation of a specific test and the subsequent transmission of that data registers as a need to sort the database, so that the interpreter is presented with only a subset of the interpreted options. The system provides the preferred combination of comments in an assembled natural language paragraph. Such a selection of the comment for a given analytic test result is driven from a priori knowledge provided through the Internet by the remote testing lab. The creation of the semi-automated test interpretation is confirmed, rejected or modified by the interposed physician test interpreter.

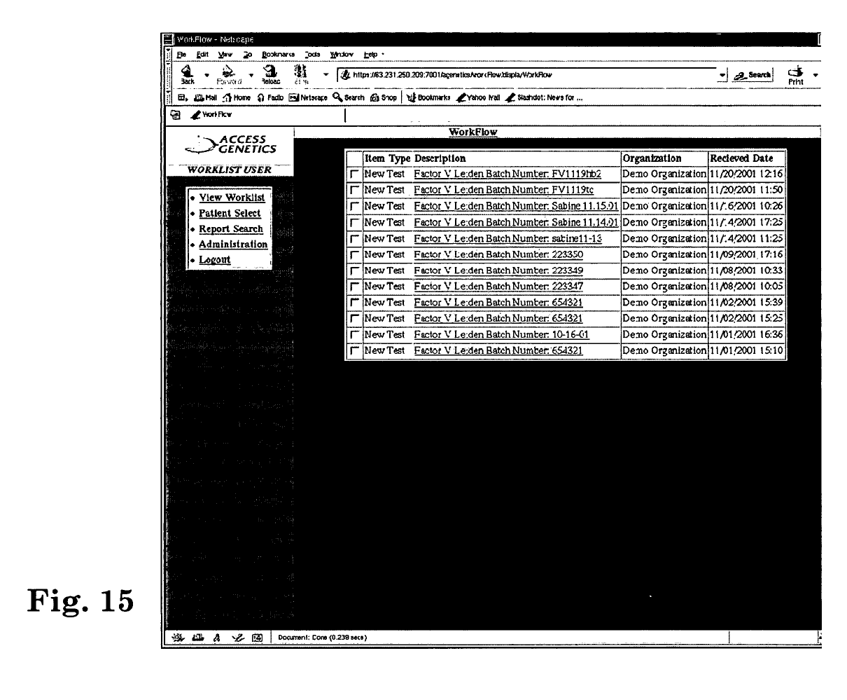

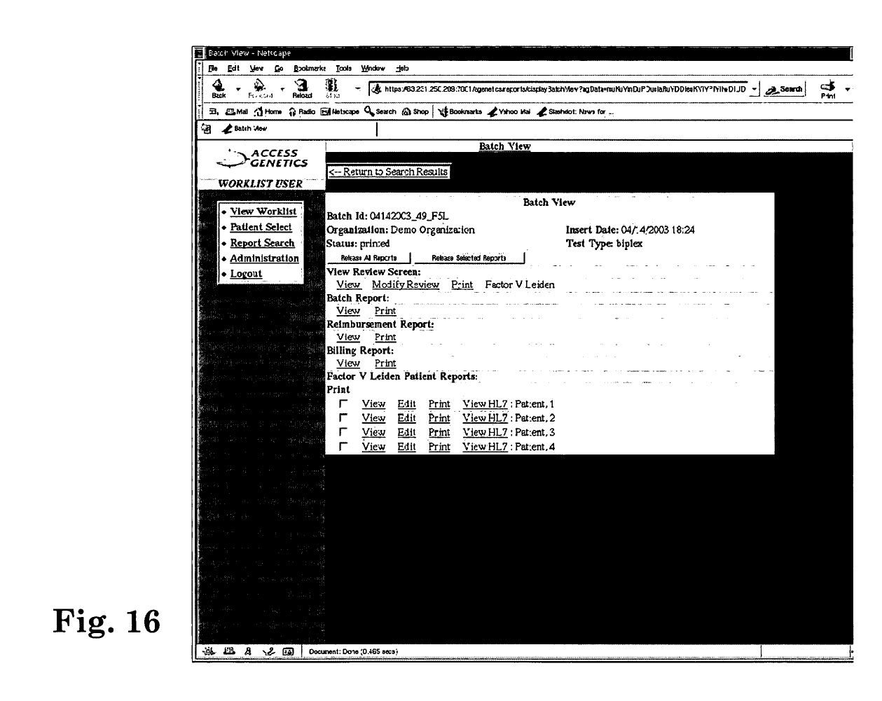

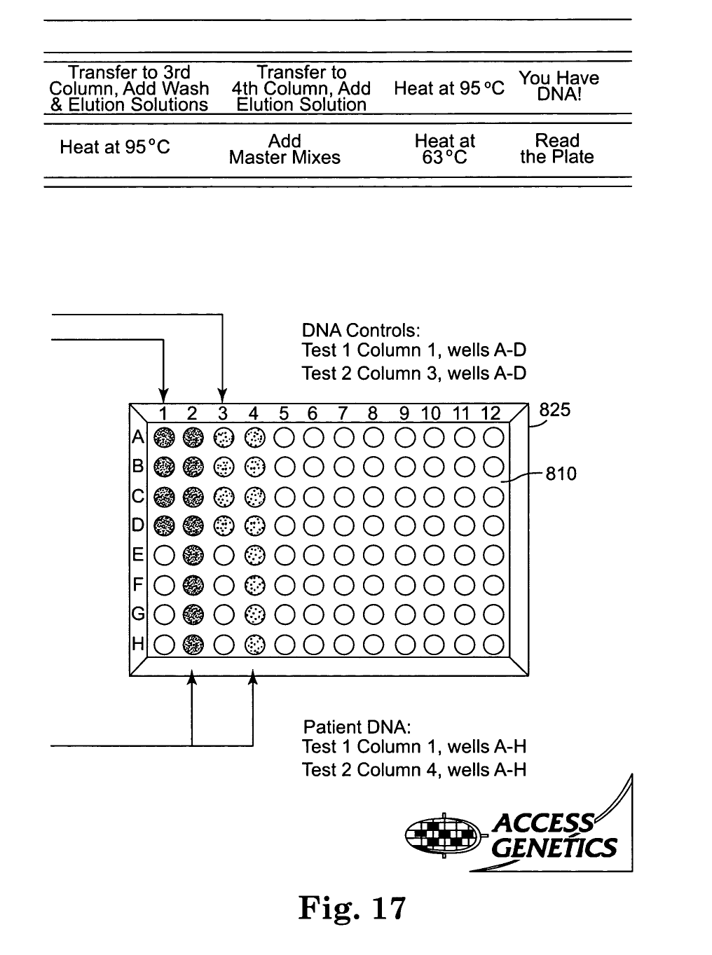

FIGS. 14-16 show possible reports created consequent to the transmission of the test data.

### Specimen Procurement

The specimens to be tested shall be obtained from the patient in an environment most convenient to the patient, such as a hospital or physician's office. The invention may include the provision of a kit, which may use existing technologies, along with all the necessary instructions and controls, to allow laboratory technicians to expertly obtain specimen samples necessary for the relevant genetic test to be performed.

### Nucleic Acid Purification

Isolation of genetic material(s) suitable for sensitive diagnostic tests requires DNA or RNA that has been separated (purified) from their cellular context and other contaminants contained in the blood, cells, tissue, or body fluid samples. Ideally, such processes are performed in a clinical laboratory in or near the clinic in which procurement of the sample from the patient occurs.

Any convenient nucleic acid purification method is suitable for use with the invention. Such a method is available from Gentra Systems, Inc. of Minneapolis, Minn. Alternative nucleic acid extraction systems or methods, such as those commercialized by Qiagen N.V., Xtrana, Inc., and others are also equivalent, as are those that perform similar results but have not yet been developed or commercialized. Specific details of the Gentra Systems, Inc. technology is described in relevant portions of the following documents (the entire contents of which are incorporated by reference), which are provided as an example of the products and processes to be used in extraction stage of the process:

U.S. Pat. No. 5,973,137 entitled “Low PH RNA Isolation Reagents, Method, and Kit”

International Patent Publication WO00066267A1 entitled “Preventing Cross-Contamination In a Multi-Well Plate”

International Patent Publication WO00049557A2 entitled “Computer-Implemented Nucleic Acid Isolation Method and Apparatus”

International Patent Publication WO09938962A3 entitled “Compositions and Methods For Using a Lysing Matrix For Isolating DNA or RNA”

International Patent Publication WO09939010A1 entitled “ELUTING Reagents, Methods and Kits For Isolating DNA or RNA”

International Patent Publication WO09913976A1 entitled “Apparatuses and Methods For Isolating Nucleic Acid”

The kit included with the invention may also include existing technologies, along with all the necessary instructions and controls, to allow laboratory technicians to expertly perform the nucleic acid purification from the specimen sample.

### Genetic Chemistry

Doctors and patients benefit by having the genetic chemistry (manipulation and amplification) portion of the genetic testing process performed on site within a clinical laboratory in or near the health care setting where the patient sample is collected and extracted (procured). On site sample procurement, extraction, amplification or some other means of genetic manipulation reduces the risks and costs associated with shipping samples to a remote location and enhances the timeliness of the results.

Possible generic chemistry techniques suitable for use with the invention include a variety of well-known, commercially available PCR (Polymerase Chain Reaction) approaches, including: (a) those known by the trademark Lightcycler from Roche Laboratories (b) those known by the trademark Labmap from Luminex Corporation; and (c) those known by the trademark Esensor from Motorola, Inc. Other suitable approaches include the micro array technology commercially available from a variety of sources, including the system known by the trademark Infiniti from AutoGenomics, Inc.

In one embodiment the preferred gene chemistry strategy employs a non-PCR approach that may be simpler for operators to use. The application of this gene chemistry to the invention involves the integration of the assembly of the reaction components, comprised of the sample control DNA or RNA admixed separately with a master reagent into a micro well incubation plate 810 all within the confines of the kit. Additionally, the system employs the use of the analytic instrument, a fluorometer, which carries out the incubation as well as serves as the interface with the Internet based controlling software. The use of the this gene chemistry includes, but is not limited to the detection of genetic test data from a solution based reaction and/or a fluorescent based reaction on a solid support such as a micro array. In each case, the data created by this gene chemistry is entered into the system and interpreted after its transport through the Internet.

Other suitable non-PCR approaches are commercially available from Third Wave Technologies, Inc. of Madison, Wis., USA under the trademark Invader and described in relevant portions of the following documents (the entire contents of which are incorporated herein by reference):

U.S. Pat. No. 6,214,545 entitled “Polymorphism Analysis By Nucleic Acid Structure Probing”

U.S. Pat. No. 6,210,880 entitled “Polymorphism Analysis By Nucleic Acid Structure Probing With Structure-Bridging Oligonucleotides”

U.S. Pat. No. 6,194,149 entitled, “Target-Dependent Reactions Using Structure-Bridging Oligonucleotides”

In addition to allowing the laboratory technicians to expertly perform the nucleic acid purification from the specimen sample, the kit provided as part of the invention also allows laboratory technicians to perform the genetic chemistry steps at their location.

### Gathering of Additional Genetic Information

In addition to the typical genetic chemistry processes described above for gene amplification or some other means of genetic manipulation, the invention could also use any of a series of analytic technologies to create raw or non-interpreted test data. These technologies may include agarose and polyacrylamide gel electrophoresis, capillary electrophoresis, fiber optic sensor devices, planar wave guide sensing devices, DNA or RNA nucleic acid micro arrays, micro mechanical biosensors, non-array based chip sensors, real-time fluorescence detectors, digital image capture, fluorometers, and the like, all according to known principles.

It should be noted that the collection of components described above is only one preferred embodiment of the invention. The full scope of the invention includes any integrated genetic testing system 200, including (without limitation) the system disclosed in U.S. Pat. No. 6,054,277 entitled “Integrated Microchip Genetic Testing System,” the entire contents of which is incorporated herein by reference.

### Integrated Kit

As illustrated in FIGS. 3-10 and 17-21B, an embodiment of an aspect of this invention is an integrated genetic test kit 800, which organizes and simplifies the technical and operational steps of most molecular genetic assays. Kit 800 may be provisioned to collect patient samples; purify the patient samples by extracting the nucleic acid from the samples; denature the purified nucleic acid and mark targeted fragments of the nucleic acid (i.e., perform the genetic chemistry) to identify characteristics of target genes; detect the genetic data output of the gene chemistry; interpret the targeted genetic data, and report the results of the test. Typically, patient sample collection and data interpretation is accomplished at a site different than the clinical test laboratory.

Kit 800 incorporates the use of various laboratory instruments and equipment, which are provided by the user of the kit. Examples of such instruments and equipment includes a computer 431, computer printer 460, microcentrifuge 822, heat blocks 821, thermometer, fluorometer 231, and multichannel expandable pipettor 808.

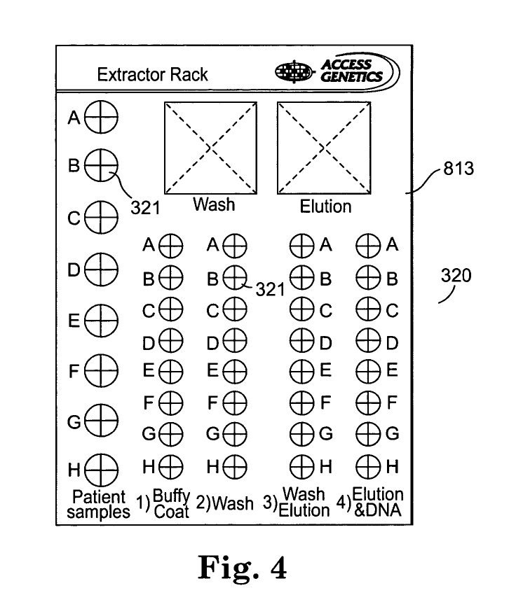

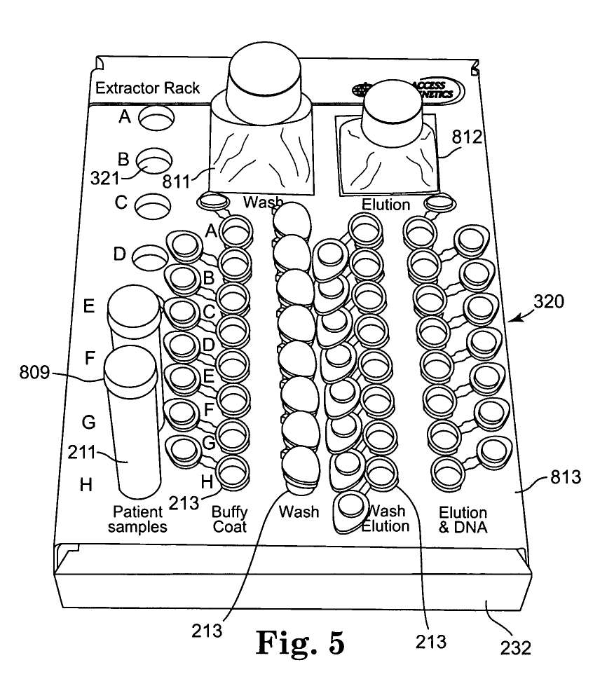

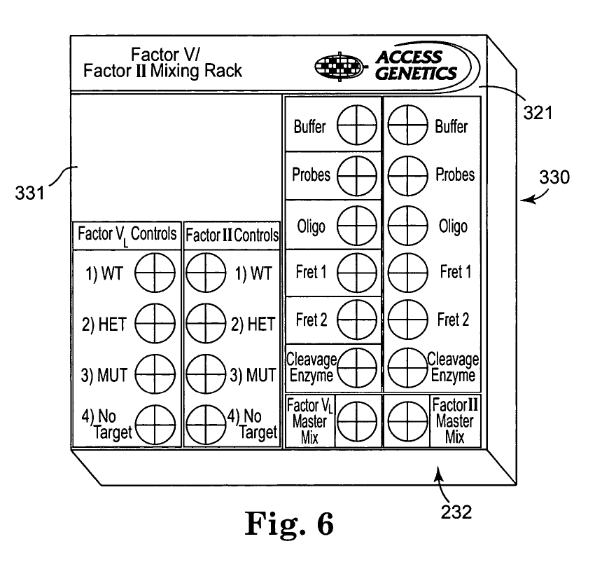

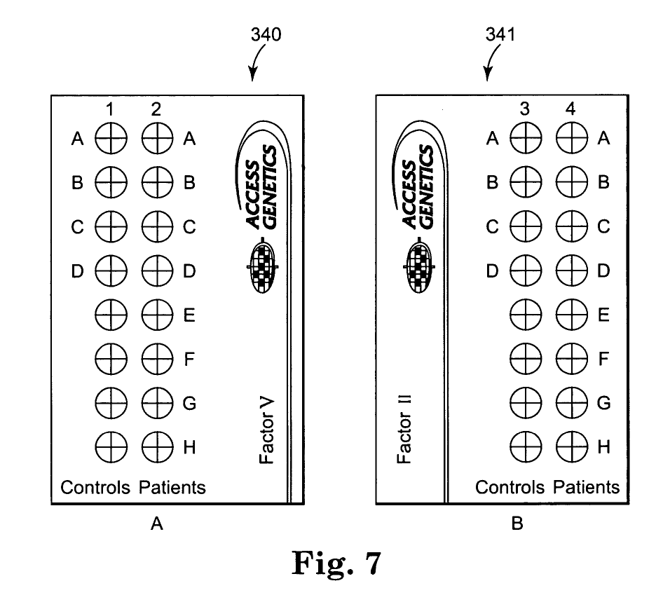

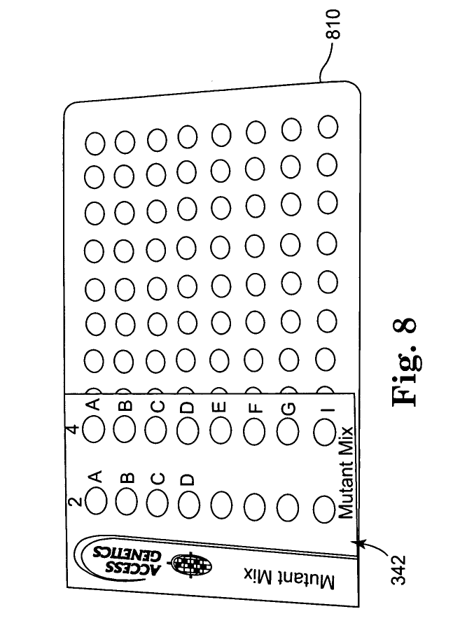

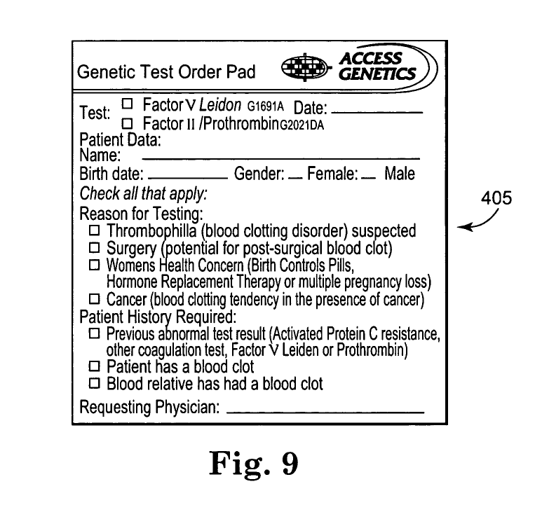

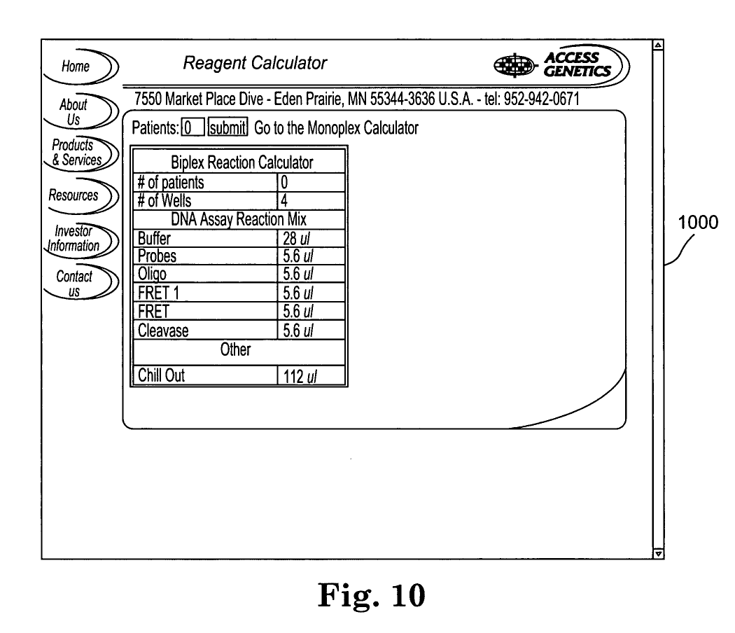

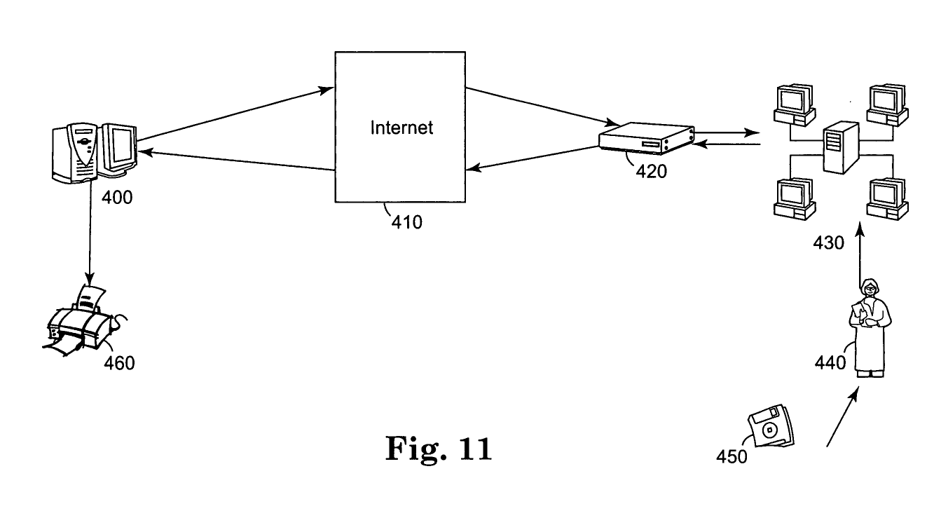

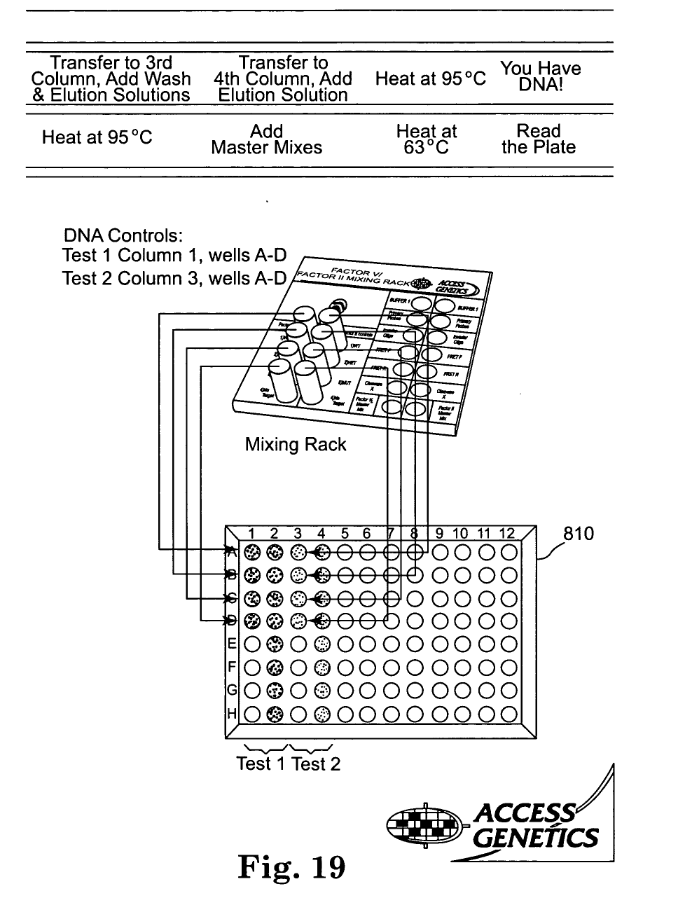

FIG. 3 illustrates test kit 800. The kit is comprised of all of the reagents, materials, and disposable supplies necessary to perform DNA or RNA based tests for detection of localized gene sequence alterations, such as detection of nucleotide substations through mutation, single nucleotide polymorphisms, and small nucleotide deletions or insertions. Test kit 800, illustrated in FIG. 3, includes kit box 801 and its packaged components. Packaged components may include one or more patient sample 210 and collection container; genetic test reagent 814 and container; reagent reservoir 824; mineral oil 218, wax 219, and their containers; labels 830 for specimens and other things; genetic test order form 405 (illustrated in FIG. 9), kit component order forms 406; suction actioned transfer pipette 212; microcentrifuge tube 213; liquid collection media 215; treated paper for cell capture 217; sequencing fixture 320, referred in this disclosure as an “extractor rack,” for extraction/purification of DNA or RNA (FIGS. 4 and 5); sequencing fixture 330, referred to in this disclosure as a “mixing rack,” for denaturing and marking the purified DNA or RNA (FIG. 6); Factor V control template guide 340 and Factor II control template guide 341 (FIGS. 7A and 7B); extraction tube 214 (FIG. 3); laboratory assay protocol in manual or electronic media 900 (FIG. 3), which details and explains each step of the genetic test process; pipette tip 803; tube snap top 809 (FIG. 5); reaction plate (microtitor) 810 (FIG. 8); skirted reaction plate base 825 (FIG. 18); wash solution and container 811 (FIG. 5); elution solution and container 812 (FIG. 5); extractor rack face plate 813 (FIG. 5); mixing rack face plate 331 (FIG. 5); a device 850 to record and store information about the components and condition of the kit; other disposable material 820; training material 902; calculator tool 1000 (FIG. 10); and other things depending upon the specific genetic test or the needs of the test technician.

The component nature of test kit 800 enables it to be provisioned from a variety of suppliers and assembled and packed before shipment to the requesting laboratory. Alternatively, test kit 800 can be shipped to the requesting laboratory and all or certain of its components packaged within kit box 801 at the laboratory site.

The component nature of test kit 800 also enables it to be provisioned precisely with the volume of reagents and disposable materials necessary to determine the gene characteristics for the particular genetic test for which the kit was ordered. Each of the components may be supplied in the quantities necessary to meet needs of the laboratory.

DNA or RNA specimens used for genetic testing are most commonly extracted from peripheral blood. However, DNA or RNA specimens may be extracted from a variety of other sources due to the unavailability of peripheral blood or because these alternative DNA or RNA specimens lend themselves to more efficient genetic testing and in certain cases provide a more reliable and accurate test result. Kit 800 is designed to extract and test DNA or RNA specimens from these alternate sources as well as from peripheral blood, and yet produce reliable, accurate, and reproducible test results. Test kit 800 is also adapted to process specimens contained in a variety of collection and transfer vessels. Preferably these vessels are graduated with markers corresponding to the optimal volume of cells needed to perform the extraction process. Examples of these transfer and collection devices are conical tubes, graduated transfer pipettes, and microcentrifuge tubes for peripheral blood. These devices are usable for collection of exfoliated cells from a Pap smear sample or the oral cavity or buccal mucosa. An embodiment of test kit 800 is optimized to accommodate multiple sources of cells suitable for testing DNA or RNA for thrombophilia genetic markers, Factor V Leiden and Prothrombin mutations. Other embodiments of test kit 800 are optimized to accommodate multiple sources of cells suitable for detection of mutation markers indicative of cystic fibrosis, human papilloma virus, gonorrhea, and chlymidia detection by analyzing DNA or RNA nucleotides for mutation markers. Often the source of specimen collection dictates the amount of specimen available for testing. The test kit is designed to accommodate these varying available sample volumes.

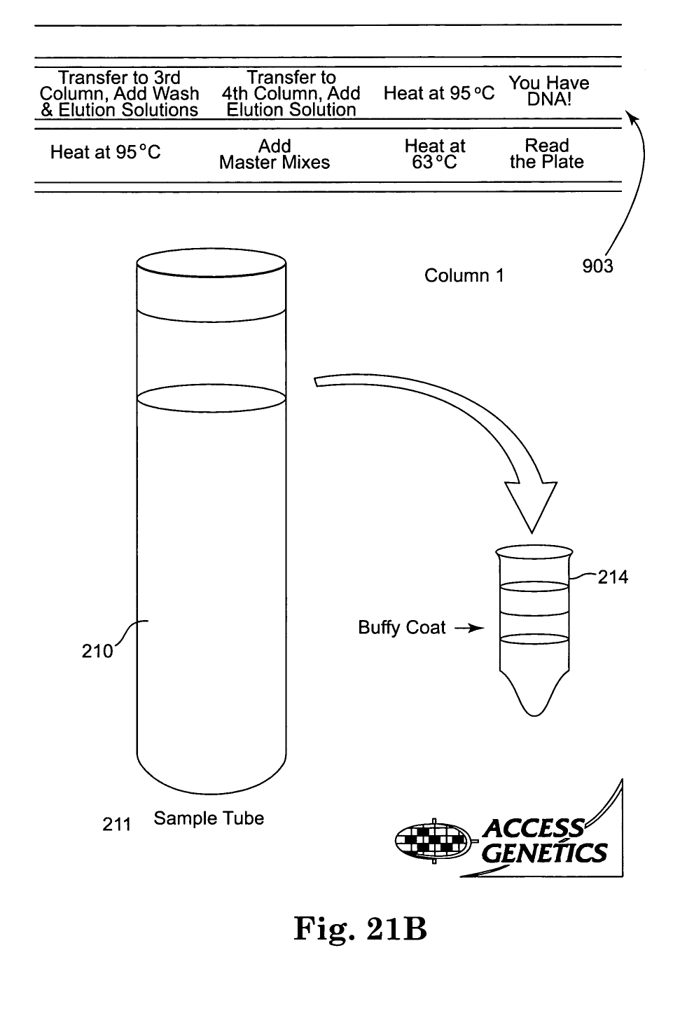

Laboratory assay protocol 900 sets forth a detailed step-by-step process for genetic testing in an integrated source document. The protocol may be in electronic or hard copy media. FIGS. 21A and 21B illustrate a set of exemplary pages of an embodiment of the protocol. The two pages shown in FIGS. 21A and 21B are a text page opposite an illustration page. Each version of protocol manual 900 follows a format whereby each step or sub step in the procedure is separately detailed (a) in text, typically on the left side page of an open book, and (b) visually by one or more illustrations, usually on the right side page of the open book.

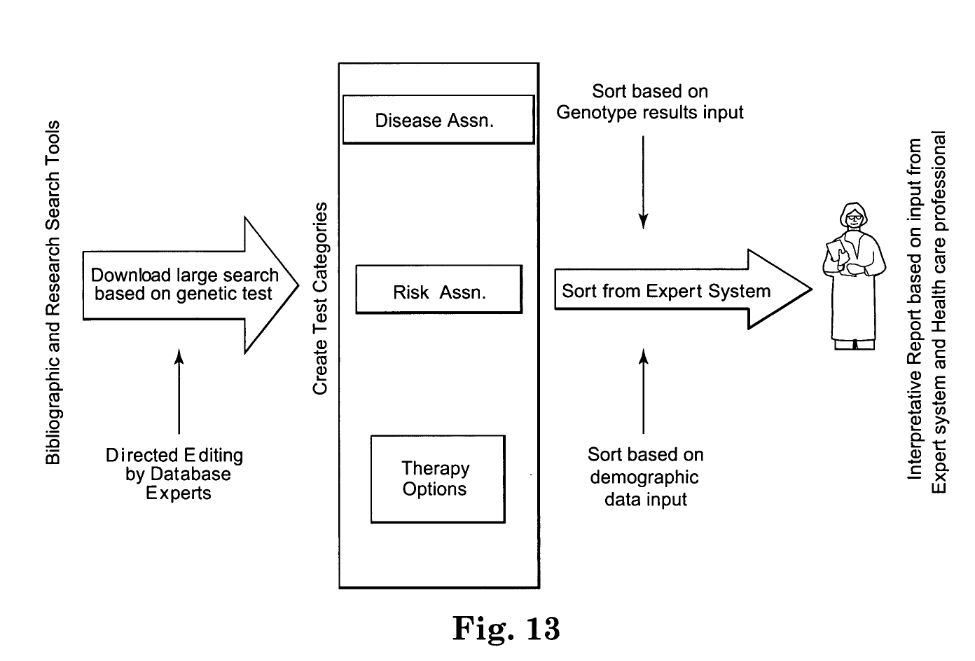

An embodiment of the protocol may include the steps of collection of patient samples, extraction of nucleic acid from the samples, and marking, and denaturing the samples. Protocol 900 combines steps from disparate multiple sources, simplifies the test procedure, improves upon the procedure, and adds steps heretofore not known. It reduces a complex process to a succinct recipe easily followed by relatively untrained or inexperienced personnel. The protocol is designed to enable the occasional genetic test person to achieve analytic results comparable to those obtained by an expert test technician. Protocol 900 also includes instructions on accessing and using calculator tool 1000 (FIG. 10) for calculation of reagent volumes used during DNA or RNA extraction, marking, and denaturing. Another calculation tool may be provided for calculating reaction times and other test process parameters. Protocol 900 includes a form 407 for entry of patient demographic data (FIG. 12) and a series of report generator forms 700 (FIGS. 14, 15, and 16), for presentation of genetic test data and its interpretation in a clear, understandable, and clinically useful format. The foregoing calculator, data forms, and report generator forms may be electronically accessible.

Protocol 900 is tailored to performance of a specific genetic test. Therefore, there will be various versions of the protocol. Versions will also vary depending upon the commercial reagent products used.

An embodiment of protocol 900 features a procedure guide bar 903 (FIGS. 21A and 21B) in the margin or header of each page. Procedure guide bar 903 lists each of the main process steps 901 in a slide bar 904 format to provide the user with an overview of each of the sequential process steps 901 of protocol 900. The two-page text and illustration format of the protocol provides the details of each separate step. The process step 901 being performed by the laboratory technician, as explained in detail on one or more sets of pages, is indicated by guide bar 903 by highlighting on guide bar 903 the short textual statement of that step with a larger or bolder font or, for example, with an illustration of a magnifying glass 905 superimposed over the step.

Labeling 830 of kit components by, for example, bar codes facilitates, in conjunction with the electronically connected supply chain, information management, and monitoring systems, (a) quality control and tracking of reagent 810 lot numbers and expiration dates, (b) restocking of kit 800 components, and (c) production of reliable test data.

Order form 405 facilitates ordering genetic tests. An order form 406 facilitates restocking of kit 800 components. Order forms 405 and 406 may be in hard copy format or electronic format for order placement over the Internet or other transmission media 400. One embodiment of order form 405 is in the form of a pad of order forms (FIG. 9). One embodiment of order forms 405 and 406 includes a series of questions to be answered by the laboratory test technician. The questions relate to patient demographic data. The questions are intended to elicit information that will assist in the determination of the appropriate genetic test and the necessary kit components. In the telemedicine model 100 of this invention, the answers trigger the genetic database and/or the expert system 450 at remote computer site 430 to assist a licensed physician to determine the indicated test based upon the test technician's information. Compliance regulations require that only a licensed physician may determine what test is indicated and place the order for the test. Order forms 405 and 406 and the associated questions provide a means to meet this compliance regulation. Transmission of the order forms 405 and 406 and patient demographic information to remote computer 430 also triggers the order fulfillment process.

Kit 800 can be used as a stand-alone apparatus for obtaining genetic test data from patient specimens 210 or the kit can be used as a component of the more comprehensive telemedicine genetic testing system 100 described to some extent in this section of the written description entitled, “Integrated Kit” and in more detail elsewhere in this written description. For example, the outputted data from fluorometer 231 or similar bioanalytic instrument (used to assay the marked genes or gene fragments) and other data may be (a) interfaced with laboratory based computer 431, connected to a secure Internet portal 400, (b) transmitted to remote computer 430, (c) stored in remote database 823, (d) interpreted by a qualified healthcare professional 440 with the assistance of expert system 450, and (e) used in preparation of an interpretive report 700 or series of reports, which will be transmitted to the laboratory. Remote computer 430 may also monitor quality control of the test procedure, track inventory of kit 800 components, and provide the laboratory with access to essential information stored on remote database 823, such as material safety data. The web-based tool illustrated in FIG. 12 facilitates transfer of patient demographic data. Patient demographic data tool 407 is a tabular display of data fields for the entry of patient demographic information such as medical and genetic history, which aids the accurate interpretation of the genetic data derived from the diagnostic test. Information in patient data tool 407 may be linked to a genetic reference database 818. In one embodiment, information entered into selected data fields in patient data tool 407 will trigger a sorting and selection function of reference information from genetic reference database 818, which may include related medical conditions and their treatment and management. Kit 800 may also contain an attached or embedded device such as an erasable programmable read-only memory 850 to store information about kit components such as material data safety, quality control, reagent expiration dates, or other such information. The kit may also contain an attached or embedded sensor to sense and store information relating to the condition of the kit during assembly, transportation, and storage, and use.

Kit box 801 streamlines the packaging, organization, storage, and performance of the genetic test steps. It also reduces the need for equipment and test specific expertise. Kit box 801 may be made of paper, cardboard, plastic, or other suitable material. It may be molded to provide a durable shipping container for the kit 800. It is labeled on its exterior with information such as the nature of its contents and purpose. The interior of kit box 801 is comprised of a series of compartments for containment of its components. One compartment 802 contains a variety of disposable things previously enumerated in this section entitled, “Integrated Kit.” Another compartment may contain reagents surrounded by an insulating material such as cellulose to maintain the reagents within a certain temperature range. Typically the number of disposable components such as tubes 213 and pipettes supplied with kit box 801 is proportionate to the number of batches of specimens to be tested. Kit box 801 is configurable for 24, 48, or 60 test batches.

Kit 800 is designed so that a hard copy manual of protocol 900 may be removably supported or affixed on the inside of kit box cover 805 or supported on a bench by a cardboard support stand provided with the kit. An embodiment of the support stand has a triangular base that holds open the covers of the protocol manual so that the pages can be turned and remain open to the desired location. Protocol manual 900 may be supported or affixed by any convenient method to the inside of the kit box cover so that when the cover is lifted the pages can be turned against the box cover. For example, the inside of cover 805 of kit box 801 may include a movable flap 806 support, which when deployed serves as a shelf for the manual. Flap 806 may be fastened by attachment means 807 to the inside of cover 805. Attachment of the flap may be accomplished by, for example, hook and loop fastener means or reusable adhesive. Flap 806 may also be a precut piece, fastened by the kit user to the inside of cover 805 using removable clips, or partially punched out of the cover material for folding into place by the user.

Another kit box 801 compartment 804 contains various specialized racks. Generally there are two such racks; the extractor rack 320 and the mixing rack 330. Racks 320 and 330 may be constructed of any material as for example paper, plastic, wood, or metal. In their most basic form they are each a rectangular body 232, each with a faceplate 813 and 331. The rack bodies are dimensioned with an area of one face to accommodate a faceplate and apertures 321 to hold various tubes, such as vacuum tubes with a stoppered top for containment of whole blood 211 and tubes 214, for containment of patient samples at various stages in processing. Racks 320 and 330 are dimensioned on another face to be of a depth that will accommodate the length of such tubes. The diameter of the apertures in the extractor rack is chosen so that the aperture at the point on the tubes that has the greatest diameter holds microcentrifuge tubes. This leaves a gap between the top of the tube and the top of the extractor rack that is in the range of 0.3 to 1.0 centimeters, which permits easy grasp of the tube when opening its top or when removing the tube from its hole. Racks 320 and 330 can be used within kit box 801 or outside of the kit box 801 as a stand-alone component. Apertures or holes 321 are sized to retain glass vacuum tubes for blood samples 211 and microcentrifuge tubes for specimen processing 213. The array of holes 321 are arranged to matingly accommodate a multichannel expandable pipettor 808, so that the center-to-center distance between holes 321 corresponds to the center-to-center distance between adjacent pipette tips 803 of pipettor 808 and the spacing of the wells in the microtitor. The distance between adjacent columns of holes 321 is related to the distance between adjacent rows of holes 321. The inter-row and inter-column distances are sized to prevent overlap of the tubes' snap tops 809 and to facilitate easy removal of individual tubes from racks 320 or 330.

Extraction of purified nucleic acid from a patient specimen 210 follows the first main step of collection of the specimens. Marking and denaturing the purified nucleic acid then follows. Thereafter, the genetic data is detected by, for example, a fluorometer. The output of the fluorometer is then analyzed, interpreted, reporting upon.

Extractor rack 320 simplifies nucleic acid extraction and purification. Its design and layout imposes (a) a step-by-step, clear sequencing, and flow of each step of the extraction process and (b) an organization of the specimens in extractor rack 320, which virtually eliminates confusion and increases test throughput. Use of extractor rack 320 in conjunction with protocol 900 further simplifies the extraction process and makes it more reliable by specification of the volumes of specimens at each of their process stages, the volumes and reagent types, timing, temperatures, and other parameters necessary for precise implementation of each of the process steps. The final step of the extraction process is the creation of a specified form and quantity of nucleic acid solution suitable for use in determination of the genetic characteristics for which the patient is tested. The extraction process is more fully described infra in the section entitled, “Integrated Test Kit Operation.”

Extractor rack 320 can be modified to accommodate various manufacturers' extraction and purification reagents. One embodiment of testing kit 800 is designed for use of the Gentra Systems, Inc. extraction reagents and its capture matrix column extraction system. The Gentra system is sold under the trademark, Generation Capture Column System. Other embodiments of testing kit 800 are designed for use of reagents from other suppliers, such as Xtrana, Inc. and Qiagen, Inc.

Extractor rack 320 retains a series of conventional or special 1.5 milli liter microcentrifuge tubes 213 in adjacent vertically oriented columns 1 through 4, illustrated in FIGS. 4 and 5, and adjacent horizontally oriented rows A through H. In one embodiment separate holes are provided in the rack to retain necessary reagents such as red blood cell lysis, nucleated cell lysis buffers, washing buffer 811, nucleic acid elution solution 812, and hydration buffers. The first column on the left-most side of extractor rack 320 is for containment of patient specimen samples 210. Samples of blood are collected in conventional evacuated stoppered glass tubes 211, which generally are of a larger diameter than is needed for microcentrifuge tubes 213 used in columns 1-4. The patient sample rows A-H are, therefore, in this embodiment of the extractor rack shown with larger diameter tube holes than shown in columns 1-4. The differences in diameters also accounts for the misalignment of patient sample 210 rows with the corresponding rows in columns 1-4. Embodiments of extractor rack 320 for use with different sized sample 210 containers will have a different misalignment or none at all. Another embodiment of the extractor rack does not have a column for patient samples 210. Spacing between the columns, the rows, and individual holes 321 within each column and row is sized to simplify the processing of specimens and to accommodate a multichannel expandable pipettor 808 for transferring and dispensing an aliquot amount of multiple samples from one tube into another tube or into reaction plate 810 without realignment of the pipette tips 803 or requiring multiple pipetting steps. Reaction plate 810 may be a microtiter. Other embodiments of rack 320 process the patient samples 210 in a right to left direction, top to bottom direction, or bottom to top direction. Each step in nucleic acid extraction and purification from an individual specimen 210 is processed horizontally from column to column. Each individual specimen 210 is confined to one row. Rack 320 accommodates multiplexing the extraction of DNA or RNA from multiple patient samples 210. Each such batch of patient DNA or RNA specimens 210 is extracted by moving one specimen at a time, horizontally to the next adjacent column, and then by moving down the row of specimens to another specimen, which in turn is advanced horizontally to the next adjacent column. This sequential process is continued until each row of patient samples is moved one column to the right and all rows of the patient samples are lined up vertically in the adjacent column. After each row of patient samples 210 in a column have been operated on in accordance with the corresponding process 900 instruction for that column, each patient sample 210 is advanced, in the manner just described, to the next successive column until all specimens 210 are in the right-most column and the extraction process is complete. A typical extractor rack 320 has eight rows, each row with an aperture 321 for retention of one of eight specimens 210. Eight specimens is the optimal number for a single technician to process. Eight specimens allows the technician to maintain a reasonable rate of flow, a low error rate, and reliable test results. An eight-specimen batch is also the batch size that best accommodates transfer of the extracted DNA or RNA from column 4 of the extractor rack 320 to reaction plate 810.

Extractor rack 320 is configured for use with Factor V Leiden and Factor II Prothrombin tests. Each step in the extraction process is performed in a single dedicated column. The left-most column on the rack is labeled, patient samples, and holds a set of 8 standard adult size stoppered vacuum tubes 211 for whole blood specimens 210. If the patient sample 210 is collected from exfoliated cells, holes 321 in the left-most column are sized for standard 1.5 mL conical tubes rather than for blood specimen vacuum tubes. Holes 321 in columns 1-4 are sized for standard 1.5 mL microcentrifuge tubes 213. Column 1 is labeled, buffy coat. Upon completion of the column 1 process step, the column 1 tubes 213 will contain the buffy coat or a cell pellet. Column 2 is labeled, wash. The buffy coat or the cell pellet, as the case may be, is washed in an aqueous based neutral salt-based buffer in this step. Column 3 is labeled, wash/elution. An additional wash step is completed in column 3 and the wash buffer is eluded from the solution. Column 4, the final column is labeled, elution and DNA. DNA is to be understood to include RNA, which may be the target intermediate material, depending upon the genetic test. The solvated DNA or RNA is eluted into a natural pH based buffer. The eluted DNA or RNA is collected in a 1.5 mL microcentrifuge tube 213. The top of extractor rack 320 has two additional holes 321 for retention of containers of wash 811 and elution 812 solutions, respectively. Wash and elution solutions are used in certain steps of the extraction process.

Denaturing and marking the purified nucleic acid is the next process step following its purification of genetic testing kit 800. Denaturing and marking the purified DNA or RNA is sometimes referred to in this written description as gene chemistry. Gene chemistry may be determined using a series of individually selected off-the-shelf reagents 810 or a series of reagents selected or formulated by a vendor such as Third Wave Technologies, Inc. of Madison, Wis., USA. Embodiments of genetic test kit 800 are configured and provisioned for use with individually selected reagents such as reagents selected or formulated by Third Wave and sold under its trademark, Invader. Other embodiments are configured and provisioned for use with other gene chemistries, including but not limited to, ligase chain reaction and strand displacement assay. The optimal technical approach is tailored for each specific gene chemistry formulation. Genetic test kit 800 includes tools, a step-by-step organization of process steps 901, reagents 810, and other components to enable a laboratory to perform the gene chemistry process on-site. Tools for enabling the technician to perform the process include mixing rack 330, a labeled face plate 813 on mixing rack 330, template guides 340 and 341; the integrated, easy to follow text and pictures of laboratory assay protocol 900; and interactive training materials 902. These tools are designed to transfer the knowledge and skill necessary to efficiently, and expertly perform the gene chemistry with the fewest steps and errors.

The combination of labeling 830 on the mixing rack faceplate 813, template guides 340 and 341 for the reaction plates, and protocol 900 provides genetic chemistry guidance not provided by reagent manufacturers. The combination controls the process in a manner that allows testing of the optimal number of specimens. It correlates the gene chemistry steps, sub steps, and procedures with the manufacturer's process information to meet or exceed all performance recommendations and requirements published by the reagent manufacturers. The combination clarifies, improves, and simplifies the heretofore confusing and incomplete test procedures, thereby increasing the probability of a successful genetic test result.

Mixing rack 330 may be constructed from any appropriate material, such as cardboard, plastic, or metal. It may be insulated or uninsulated. It comprises a rack body 232 and a faceplate 331 and contains a matrix of spaced and sized holes 321. Mixing rack 330 may contain a number of holes that will be usable for a variety of tests, some of which tests may require fewer holes than provided on the mixing rack. Such a standard mixing rack may be preferable from an inventory control point of view. Each such standard mixing rack is labeled with a faceplate 331 designed for a specific test, in some cases leaving some holes unlabeled and unused. Or, mixing racks may be provided that are tailored for a single specific test with only the specific number and location of holes needed for such specific test. Faceplate 331 and template guides 340 and 341 may be made of cardboard, paper, plastic or other printable material. Faceplates 331 and 813 are removable from the racks and may be replaced with a different faceplate to correspond with a different test.

An embodiment of the gene chemistry step for the Factor V Leiden and the Factor II genetic test kit 800 is performed using reagents 331 supplied by Third Wave Technologies, Inc., mixing rack 330, face plate 813, and template guides 340 and 341 as illustrated in FIGS. 7A and 7B. The gene chemistry step for the HPV genetic test kit is performed using Polymerase Chain Reaction reagents supplied by Hoffman La Roche. The mixing rack 330, faceplate and template guides are specifically designed for the use of the PCR method. The purified nucleic acid is combined in reaction plate 810 with the control samples and master unit from the mixing rack.

Mixing rack 330 and template guides 340 and 341 index DNA or RNA controls 816 and patient samples 210 and guide the use of master mixes 815. Template guides 340 and 341 contain holes in columns and rows to guide each tip of a single or multichannel pipette 808 into the wells of a 96 well microtiter 810 to simplify the addition of multiple liquid reagents 814, samples, or other materials. The holes 321 in template guides 340 and 341 correspond to center-to center distance between the holes in microtiter plate 810. Guide templates 340 and 341 are two-sided with labeling and numbering on both sides of FIG. 8 illustrates a reaction plate 340 with another template guide 342 in position on the left side of reaction plate 340. Template guide 342 is for use with the mutant mix reaction. It too, is two sided. The other side guides the wild type reaction. Yet another two sided template 343 guides the reaction condition with no enzyme added on one side and with enzyme added on the other side. The labeling on each side of templates 340, 341, 342, and 342 may be asymmetric with regard to the alignment of the holes in microtiter plate 340. Template guides 340, 341, 342, and 342 may also have reference marks to guide alignment of the template with the edges of the reaction plate 340. The purpose of these fiducial points may be to guide each of patient samples 210 into the respective reaction wells 819 or to permit the combination of multiple, separate tests for the same patient sample 210 in a single reaction plate 340.

Master mixes 815 and control samples 816 are essential to the non-PCR approach. When used with the non-PCR process, mixing rack 330 facilitates organization of DNA or RNA control samples 816. After DNA or RNA control samples 816, which are inserted into reaction plate 810.

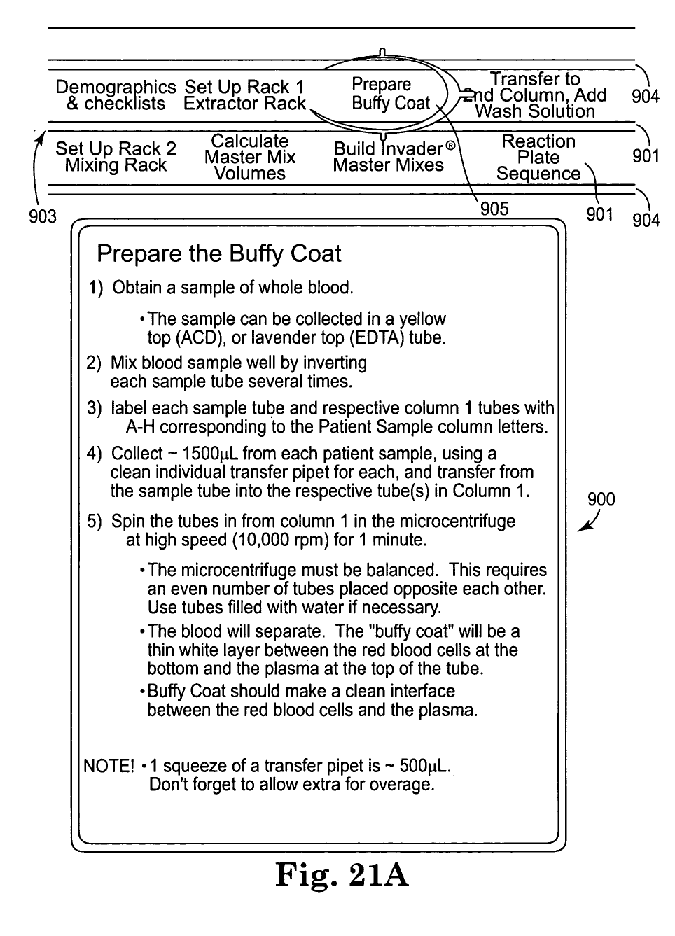

Mixing rack 330 sequences reagents 814 which become constituents of a common reagent mix, referred to in this invention as master mix 815 formulation. The individual chemicals or reagents to carry out a gene chemistry reaction are organized within the rack in microcentrifuge tubes 213 retained in holes 321 for each reagent. The mixing of these reagents, according to protocol 900, occurs by adding reagents 814 from each individual reagent container to a common vessel for assembly of master mixes 815, as illustrated in FIG. 20. Six tubes 213 of reagents are included in master mix 815. Each reagent is different. Each hole 321 in mixing rack 330 is labeled to correspond to labeling on the reagent tubes and the reagent's function in the gene chemistry protocol 900. Each reagent 814 tube is placed in its respectively labeled hole 321 in mixing rack 330 for use during the master mix 815 creation process.

The six different reagents for both Factor V Leiden and Factor II genetic tests are generically buffer, probe, oligonucleotide, a first FRET (fluorescent resonance energy transfer), a second FRET, and a thermostable endonucleolytic restriction enzyme for cleaving nucleic acid at a specific site for isolation of a nucleotide sequence encoding for the target protein relevant to the blood factor under study. The first and second FRETs are respectively the fluorochrome FAM and Texas Red. Both fluorochromes are available from Third Wave Technologies, Inc. Third Wave sells them under the Invader trademark. Third Wave's Invader technology is based on the recognition of specific 3 dimensional structures in composite DNA, which are assembled only when a precise sequence is present. The Invader enzyme, Cleavase, may be used to fragment the 3D structure generating a fluorescent-labeled flap of DNA. The result is a numerical value that can be presented as a ratio and compared with the ratios of established controls to determine the genotype. The Third Wave Invader assay product relies upon the specificity of its cleavage enzymes, which recognize only the invasive complex, permitting discrimination of single base changes. In the case of a single base invasion, i.e., the formation of an invasive complex, there is cleavage and a fluorescent signal is detected. If an invasive complex is not formed, cleavage does not occur and a fluorescent signal is not detected.

There are two columns on the right side of mixing rack 330. The left-most of the two columns retains the reagents and master mix 815 for Factor V Leiden test. The right-most of the two columns retains the reagents and master mix for the Factor II test. Holes in each column are labeled from top to bottom with the names of the reagents and the master mix for the respective two tests. Mixing rack 330 is designed for processing one patient sample 210 at a time. A designated volume of buffer reagent is pipetted out of its respective tube, which is located in one of the two right-most columns of the mixing rack, depending upon which master mix is being compounded. The specified volume of the buffer reagent is pipetted into the master mix tube located in the same column from which the reagent came. In the same manner, the primary probe reagent is next added to the respective master mix tube. Then the oligonucleotide, the first FRET, the second FRET, and the thermostable endonucleolytic restriction enzyme reagents are added to the master mix tube in that order. The workflow can be in a bottom to top, left to right, or right to left direction instead of the top to bottom direction described above. It is the organization of the work flow in an ordered efficient manner that is paramount. The ordered manner reduces process time and errors.

The boxed in area 322 surrounding each of holes 321 in mixing rack 330 is identified both with a written notation and also with a color to indicate which reagent belongs in which hole 321. The boxed in areas 322 increase from a pale shade of a color to an ever more intense color to correlate with the process steps set forth in laboratory assay protocol 900 and training materials 902. The test technician is instructed to proceed from pale to intensely colored boxes 322 to create master mixes 815. All full intensity color boxes 322 contain fluids that will be transferred to reaction plate 810 for performance of the reaction plate process sequence. The column on mixing rack 330 for the Factor V Leiden test is colored differently than the column for the Factor II test. This color coding schema for indication of different test processes reduces technician mistakes and decreases the time to complete the mixing process.

Mixing racks for each different test will have differing arrangements for the various reagent tubes used to create a master mix 815 for each test, as well as color coding schemes.

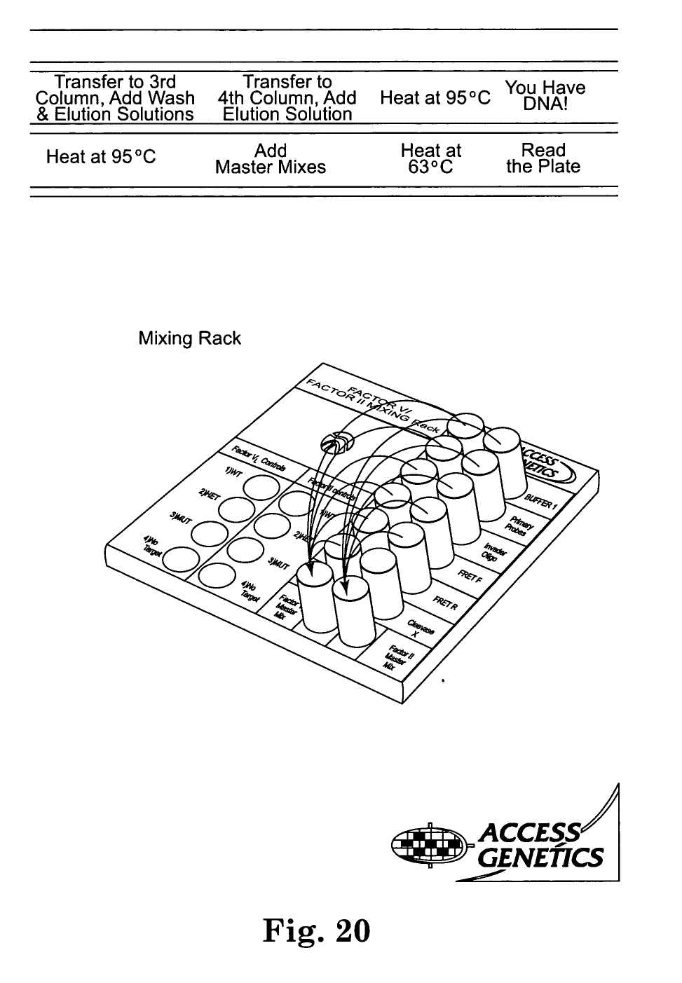

A mixing rack 330 arrangement similar to that for reagents 814 and master mixes 815 is adopted as the arrangement for the DNA or RNA control tubes. The DNA or RNA control tubes are retained in the two left-most columns of mixing rack 330. Control tubes for Factor V Leiden are in a column that is the same color as is the color of master mix box 322 for Factor V Leiden. Likewise, the control tubes for Factor II are in a column that is the same color as the color of master mix box 322 for Factor II. The placement of control tubes 816 in mixing rack 330 corresponds to the orientation of wells 819 of reaction plate 810, in which the contents of the control tubes will be sequentially added, as shown in FIG. 19. The spacing between control tubes in mixing rack 330 and wells 819 are the same to facilitate movement of the contents of control tubes to the wells 819 using a multichannel pipettor 808. With this arrangement the center-to-center distance between adjacent pipette tips 803 of the pipettor need not be changed when transferring control tube contents. Mixing rack 330 may be used as a stand-alone device or for use in connection with integrated kit 800. When used in connection with the integrated kit, the user is guided and aided by placement, labeling, and orientation of the respective tubes and by the clearly stated process steps described in protocol 900.

As with the extraction process, the determination of reagent volumes and other process parameters for the mixing process are aided by tools such as calculator 1000, which may be in hard copy or electronic form. Laboratory assay protocol 900 may direct the test technician to use calculator tool 1000 to calculate reagent volumes to make up master mix 815. An embodiment of such a calculator tool is illustrated in FIG. 11. Entry into calculator 1000 of the number of specimens in the test batch triggers the programmed calculator to calculate the necessary volume of each reagent required to make a master mix 815.

Test kit 800 or racks 320 or 330 may contain holes for retaining additional additives to the gene chemistry process, including non-miscible oils such as mineral oil, waxes, or other such materials that prevent evaporation of heated reactants.

Another component of genetic test kit 800 is reaction plate 810. Reaction plate 810 may also comprise template guides 341-343 having a series of small holes that correspond to reaction wells 819 in reaction plate 810. The templates are comprised of a planar sheet of paper, plastic, or other printable material. After the gene chemistry step is completed the (a) purified nucleic acid, (b) master mixes 815 and any other materials optionally added to the master mixes, and (c) the controls, are transferred to reaction plate 810.

Combination of the various volumes of the extracted nucleic acid and reagents 814 into each specific reaction well 819 of reaction plate 810 would be a time consuming and painstaking process were it not for handheld multichannel pipette 808. Pipette 808 is a highly accurate and precisely calibrated instrument. A multichannel pipette is commercially available from Apogent Discoveries, Inc. as well as other sources. Pipette 808 is well suited for accurate pipetting of multiple small volumes from the various sized tubes used in DNA or RNA extraction, master mix preparation, and transfer into small, precisely positioned reaction wells 819.

In one embodiment of reaction plate 810, a mylar laminated paper template card is dimensioned to fit plate 810 as illustrated in FIG. 7. Template 340 helps the technician avoid placement of the wrong control or patient sample in a reaction well 819. Another template 343 indicates whether or not an enzyme is present. Another template guide 342, as previously described, is labeled on one side to correspond to one type of gene chemistry reaction, such as the reaction for the wild type (normal genetic allele) of a particular genetic marker. The opposite side of template 342 is labeled for the mutant (alternative genetic allele) for the same genetic marker. Therefore, to perform the desired gene chemistry reaction for a specific allele, the operator merely selects the appropriate side of guide 342 and positions that side face-up against one particular edge of the underlying reaction plate. The holes in guide 342 permit proper alignment of wells 819 with the samples for the desired gene chemistry reaction. When the opposite side of guide 342 is used, the holes then align to the wells designated for the alternative reactions.

### Integrated Test Kit Operation

Laboratory assay protocol 900 is integral to genetic test kit 800. It is the process by which the clinical technician is guided to use the assemblage of kit 800 in performance of the genetic tests for which it is configured. The protocol and the test kit are an integrated unit. They are designed to work hand-in-hand; each enhancing the organization of the other. Protocol 900 may reside in any media. It may be in a paper format accompanying kit 800 or in an electronic format residing in the laboratory computer 431 or in the remote computer 430 for downloading to the laboratory computer via the Internet or some other data transmission system 400. One embodiment of protocol 900 is presented in the paragraphs that follow in this “Integrated Test Kit Operation” subsection. This embodiment of protocol 900 includes the process steps for conducting the Factor V Leiden/Factor II Prothrombin Thrombophilla set of genetic tests. Other embodiments of protocol 900 vary to encompass other genetic tests. One common factor in each embodiment is that protocol 900 guides the technician through each of the various genetic test processes in combination with kit 800, configured for a specific test. Representative portions of protocol 900 for the Factor V Leiden and Factor II tests are illustrated in FIGS. 17 to 21B.

Prior to beginning the genetic test process, patient samples must be collected. Usually these are collected at a different site than where the genetic test is performed.

Cells for genetic testing are most often obtained from peripheral blood. Cells from scraped or exfoliated tissue are also a source as is tissue biopsy material. Test kit 800 is equipped to use cells collected from any of these sources, although different procedures must be used to prepare the patient samples for testing. Cells collected by each of these processes may be used for extraction of nucleic acid to test for mutations indicative of the genetic markers, Factor V Leiden and Factor II.

One method for preparation of collected blood, exfoliated cells, or tissue comprises depositing the specimen on a solid support material, such as a specially treated paper 217. The treated paper captures the intact cells and removes the fluid from the specimen. The cells are then washed from the surface of the paper into a vessel. Such specially treated paper is sold under the trademark, FTA. This collection process is often used for forensic testing.

Another method for preparation of exfoliated or scraped cells uses liquid collection media 215. The liquid collection media 215 collects and fixes the exfoliated or scraped cells, which are in mixture with an alcohol based preservative. Exfoliated cells may be collected from the mouth upon rinsing with a water-based mouthwash. Both the specially treated paper and the liquid collection media process are designed to optimize the number or volume of cells necessary to extract a high volume of nucleic acid. The liquid collection media process uses any of a variety of commercially available liquid collection media 215, such as media supplied by Cytec or SurePath.

Peripheral blood is anticoagulated with the usual preservatives such as acidified citrate dextrose (ACD), ethylenediaminetetraacetic acid (EDTA), or sodium heparin.

In the telemedicine embodiment of this invention, the process begins by opening a web-based file. The user enters his/her name, the number of patient samples, and the test type. The system automatically provides the user with a batch number. The user then enters patient and demographic information.

The heat blocks are pre-heated to the temperature specified for the genetic test type. When performing the Factor V Leiden and Factor II tests the temperature is 95° C. for a 3 block analog heat block and 63° C. for a 2 block digital heat block. A fluorometer, microcentrifuge, microcentrifuge timer, and laboratory computer and printer are also used to conduct the process of this invention.

The process of extraction of nucleic acid from whole blood patient samples includes preparation of a buffy coat. Each blood sample tube is labeled with the letters, A-H. The samples are placed in rows of the patient sample column of the extractor rack. Each row is labeled with the corresponding letter of the patient sample.

An aliquot amount of the anticoagulated whole blood is pipetted from the sample tube into a suction actioned transfer pipette 212, inscribed with measured “hash marks.” The aliquot amount of whole blood is an amount that ensures the extraction of enough DNA or RNA to perform the gene chemistry in accordance with this invention. The pipetted whole blood is then transferred to a microcentrifuge tube 213 and the tube is placed an aperture in the same row as the sample in a column 1, labeled buffy coat. Each of the tubes in column 1 is labeled respectively with the letters A-H. A new transfer pipette is used for each patient blood sample. Exfoliated or scraped cells are also transferred to a microcentrifuge tube and placed in column 1. The tubes in column 1 are then spun in the microcentrifuge until the blood separates into plasma at the top, a thin white layer of buffy coat below the plasma, and red blood cells at the bottom. A suitable buffy coat makes a clean interface between the red blood cells and the plasma. The exfoliated or scraped cells are also spun. They are spun until they become pelletized 216. The microcentrifuged column 1 tubes are returned to their respectively labeled locations in the extractor rack. The buffy coat is next added to the tubes in column 2, which column is to the immediate right of buffy coat column 1. Column 2 is labeled, “wash.” Each of the tubes in column 2 is labeled respectively with the letters A-H. The buffy coat from each patient sample is transferred, one at a time, from each tube in buffy coat column 1 to the correspondingly labeled tubes (with a white filter basket in the tube) in wash column 2. A clean transfer pipette is used for transferring each buffy coat. The full volume of the buffy coat is then aspirated with a swirling motion and dispensed evenly across the top of the filter basket without overloading the filter matrix. Overloading may lead to low DNA or RNA yield due to incomplete cell lysis. An aliquot amount of wash solution is added to the center of each filter basket. A trough is labeled “wash” and filled with an aliquot amount of wash solution. The tubes are spun in the microcentrifuge until red tinged fluid ceases to filter through each filter basket.

The filter baskets are next transferred to the third column on the extractor rack. The third column is labeled, add wash and elution. First the tubes in wash/elution column 3 are labeled, A-H. The filter baskets from column 2 tubes are transferred to the correspondingly labeled tubes in column 3. An aliquot amount of wash solution is added. Column 3 tubes are spun in the microcentrifuge and an aliquot amount of elution solution is then added to the filter basket in the column 3 tubes to facilitate separation. A second trough is labeled, elution, and an aliquot amount of elution solution is transferred into it. Column 3 tubes are spun in the microcentrifuge again. Pink fluid will filter through the baskets and collect in the bottom of the tubes.

The filter baskets are now transferred into column 4, elution solution is added, column 4 tubes are heated, and DNA or RNA is isolated. This step is accomplished by a series of sub steps, the first one of which is to label each tube in column 4, A-H. The filter baskets from column 3 are transferred into the correspondingly labeled column 4 tubes. An aliquot amount of elution solution is added to each column 4 tubes. Column 4 tubes are fully inserted into the larger holes of the dry heat block. The filter baskets must be completely surrounded by the holes within the heat block. The tubes are incubated. Disposable materials are discarded throughout the process. However, the original patient samples are retained for additional tests, if necessary. The tubes are immediately centrifuged after their removal from the heat block. The tubes are removed from the microcentrifuge and put back into their positions in column 4 of the extractor rack. The volume of liquid in the bottom of the tube is nearly clear, but a slight red tinge is acceptable. The volume contains genomic. DNA or RNA. The filter baskets are discarded. The column 4 tubes remain in column 4 until the mixing process is completed. Upon completion of the mixing process the nucleic acid in the column 4 tubes are transferred into the appropriate reaction plate wells. The DNA or RNA tubes can be stored at 4 degrees centigrade and the mixing step may be completed the next day, if necessary. The useful life of DNA or RNA is no more than 2 months.

The genetic chemistry process begins with the mixing step. First, the frozen reagents are thawed and placed in the mixing rack. The reagents used in this step are (a) a buffer, (b) probes, (c) oligo, (d) a first Fret, (e) a second Fret, and (f) a thermostable endonucleolytic restriction enzyme for cleaving nucleic acid at a specific site for isolation of a nucleotide sequence encoding for the target protein relevant to the blood factor under study. The reagents are shipped separately and stored at 20° C. Master mix tubes are labeled with the name of the test being performed. The clear, labeled master mix tubes are inserted into the correspondingly labeled master mix hole in mixing rack. Each thawed reagent tube is tipped and flicked to mix thoroughly. All tubes are centrifuged (including the control tubes). Each tube is then replaced into its respective hole in the mixing rack. The volume of each reagent used to make up the master mix is the product of 1.25 multiplied by the sum of the number of patient samples plus the four controls multiplied by (a) 5 micro liters for the DNA or RNA reaction buffer, (b) 1 micro liter for the primary probes, (c) 1 micro liter for the oligonucleotide, (d) 1 micro liter for the first FRET, (e) 1 micro liter for the second FRET, and (f) 1 micro liter for the cleavage enzyme. Alternatively, the volume of each reagent used to make up the master mix may be determined using the reagent calculator, selecting the test type, entering the number of patients in the batch, and reading each reagent volume from the calculator.

Mixing of the master mix begins by adding each reagent, one-at-a-time, to the reagent's designated master mix tube. The reagents are added by using one pipette tip at a time for each reagent. Each tip is disposed of before pipetting the next reagent. This part of the process starts at the top of the column of tubes for a particular test and works down the column sequentially from lighter to darker color. Each reagent is added to the master mix tube to make the collective master mix solution. The master mix tubes must be tipped and flicked to mix thoroughly. The master mix is then spun on pulse and returned to its place in the mixing rack.

Transfer of the master mix, the control samples, and the purified nucleic acid to the reaction plate is the next step of the protocol. The reaction plate may be tailored from a 96 well microtitor by cutting the microtitor plate so that it includes only the number of columns needed for the controls and patient samples. The reaction plate is labeled with the date, the laboratory technician's initials, and a dot in the upper left corner to indicate proper plate orientation. The reaction plate is placed into the skirted base for stability with the dot in the upper left corner for orientation. An aliquot amount of each control solution is pipetted from the mixing rack to reaction wells A-D in columns 1 and 3, as shown in FIG. 19. All four control solutions are dispensed into the reaction plate simultaneously. Control solutions from rows 1, 2, 3 and 4 of the mixing rack must be oriented from top to bottom in the reaction plate. Reaction wells E-H, below the control solution in column 1, are left empty. Next, the patient's nucleic acid from column 4 of the extractor rack is transferred into the corresponding A-H wells in columns 2 and 4 of the reaction plate. If patient nucleic acid was extracted 1 or more days prior to performance of the genetic test, the tubes must be thoroughly mixed and then centrifuged before adding it to the reaction plate. When two tests are performed on the same reaction plate as described in this paragraph and in the Figures, the respective control solutions for the second test must be transferred into column 3 and the same patient nucleic acid sample must be transferred into column 4 of the reaction plate. The patient nucleic acid must be orientated top to bottom, A-H. An aliquot amount of mineral oil is dispensed on top of both the control solutions and the patient nucleic acid in each reaction plate well, using a new tip for each well. An aliquot amount of mineral oil is dispensed into a labeled trough. The reaction plate is then heated by a heat block that is temperature stabilized at a temperature specified for the specific test underway, which is 95° C. for 7 minutes±2 minutes for the Factor V and Factor II tests. The heat block's temperature must between a minimum of 93° C. and a maximum of 99° C. After heating for a specified time period, the reaction plate is moved from the 95° C. heat block to a 63° C. heat block. While the plate is in the 63° C. heat block, master mix is added to the reaction plate. When two tests are performed, an aliquot amount of master mix is dispensed into each of the 4 control wells in columns 1 and 3, rows A-D, of the reaction plate and each of the patient wells in columns 2 and 4. The plate stays in the heat block during this process. This process must be done one well at a time with a new pipette tip for each well. The reaction plate wells are kept track of by arranging the pipette tips in their box in the same configuration as the control and patient layout of the reaction plate. The pipette tip is pushed through the mineral oil to the bottom of the well and then dispensed. The pipette trigger is held until the tip is completely withdrawn to avoid re-aspiration of liquid, which could potentially jeopardize the results. The reaction plate is at this point heated to 63° C. for 4 hours±10 minutes to incubate. All volume levels in the reaction plate should be the same. Any bubbles present should float to the top during incubation. The reaction plate is returned to the 63° C. heat block. The unused reagents are refrozen at −20° C. The leftover patient nucleic acid is stored in the refrigerator at 4° C. for up to two months, in case additional tests should be required. After four hours the reaction plate may be read.

The final step in the genetic test process is to read the genetic test data, i.e., detect the data, and transmit it to a site for interpretation and report generation. The reading step is initiated by opening a genetic test kit file folder on a computer. The reaction plate is placed in the fluorometer with the black dot in the upper left corner. Upon entry in the file folder of a password and the batch number for the test, a flourometer is triggered to read the reaction plate. The results are automatically sent to the remote computer for interpretation and report generation. Pending receipt of the search results, it is recommended that the reaction plate be inserted into the skirted base, covered with foil, and stored in the refrigerator until a test report is generated for each patient. The test reports are downloaded when completed.

The protocol specifies the various parameters for each step of the test performed using the integrated test kit. These parameters differ depending upon the test conducted. Certain of these parameters include temperatures and temperature tolerances; heating and cooling durations; storage temperatures and maximum storage duration; thawing times of samples, reagents, and controls; the rpm when centrifuging and the duration thereof; and the volumes and concentrations of reagents, samples, control samples, master mixes, and mineral oil or other evaporation impeding substance. The protocol specifies that a multichannel pipettor device be used for certain transfer operations. The pipettor device is encouraged or required, as the case may be, to increase the rate of test throughput, ensure accuracy of volumes transferred, and significantly reduce error rates.

### Data Collection

After the raw or non-interpreted genetic data and any necessary enhancements to increase clarity are generated, they are then gathered at the remote location in any convenient manner by data collection system 300. In addition to scientific clinical data, data collection system 300 also is preferably supplied with or gathers relevant demographic data about the patient. Based on the genetic test performed, the data collection system 300 gathers from the remote location, to the extent available, patient demographic data that enhances interpretation of the analytic data gathered at the remote site, analyzed in the central data analysis/interpretation system 600, and/or reported in the form of report data 700. The relevant demographic data about the patient report could consist of, but is not limited to, patient identifier, gender, age, clinical history, billing information, and other correlative information about the patient including written or numerical identifiers, current and historic physical characterizations of the patient's state of health, past laboratory, physical exam or specialized studies and commentary from qualified medical professionals.

An aspect of this invention is that patient demographic data may be parsed, extracted or in some manner derived from electronic databases at or associated with the site where the patient interface occurs or at the site where the technical aspects of the test is performed. This process of deriving patient demographic information may involve information systems outside the system.

Additionally, patient demographic information may be entered into the system by the physician, nurse or laboratory technologist based on the responses provided on the “Genetic Test Request Pad”. The Genetic Test Request Pad is a media to convenient transcribe information pertinent to the interpretation of the genetic tests into the system. The information collected varies for every test, but may include such facts as the date of birth, gender, listed medical conditions, responses to specific questions relevant to the particular test at hand, and any additional laboratory data that may precede the application of the genetic test. For a single or combination of tests offered, the genetic test request pad is customized to include questions and demographic data pertinent to the interpretation of that selection of tests.

Within the system, the preferred embodiment for gathering demographic data is a simple spreadsheet with fields that permit entry of the responses to the questions on the Genetic Test Request Pad. This may involve the entry of data by any suitable technique, such as voice command, typing, touch screen, or by selection of electronic buttons or menus.

Demographic patient data may be used in any of the following ways. First, the data may be used to create a test identification number that links a certain patient with their respective analytic results. Second, the selection of demographic data may be used to sort interpretative information in the expert database 650. Third, the data may be used to organize the placement of samples on the reaction plate.

The preferred embodiment for the data collection system uses computer networking techniques and systems to electronically gather data with as little human involvement as possible. The derivation of the analytic data involves the creation of a software interface to an analog data source through a connected computer, which in turn is connected to a central server at a site distant to the remote laboratory through the Internet. Through this interface the system can address the remote laboratory without the aid of operator actions. One embodiment of the invention includes the warm-up of the fluorometer and the ability to address that instrument to determine if reaction plate incubation is ongoing. This is achieved by means of a software derived “scout” that queries the instrument for a variety of its functions. The software permits the periodic cessation of the incubation and subsequent read of the reaction plate to determine if that operation is complete or incomplete. The software interprets the control in each reaction plate to determine if an adequate level of fluorescence signal has been created. If the reaction is complete, then the system prompts the interpreter to read the plate, otherwise the plate is returned to the incubation mode, and the process is repeated later in time. The software is designed to control of the assay operations such as control of heating sources, mechanical movement of the plate and or plate holder, mechanical agitation of reactions and the operation of the readout functions of the machine. The interface also allows us to acquire and process the numeric results after a batch of tests are read.

The capabilities to determine what information to gather and how such information is gathered by the data collection system 300 is an important embodiment of the invention.

### Verification of the Genetic Testing Data

In addition to gathering data at the remote location, depending on the preferences and capabilities of the remote location, the data collection system 300 may also include the capability to provide mathematical representations and/or transformations of raw and other data, for quality control or other purposes, prior to transmission to the central location. Specifically, the data collection system 300 may automatically perform a preliminary review of the data gathered to determine the existence of information necessary for the central location to complete the genetic test and generate useful reports and other feedback. If such review determines insufficient or inconsistent data has been collected, the data collection system 300 generates a report for the remote location user identifying such issues along with recommendations to correct the problem. In addition to being located at the data collection system 300, such capabilities could also be provided in central data analysis/interpretation system 600.

The invention may include verification of the completion of the amplified genetic testing data produced on-site, with transmission of such data and specific patient demographic information through a proprietary hardware and software system to a central pool of experts in diagnosis and genetics. This enhances the quality and the value of the test results and information provided for both the doctor and the patient. One possible embodiment of this aspect of the system comprises the generation of an e-mail or voicemail prompt to the designated expert informing them that test results are available for review. This prompt may be automatically generated when a batch of test reactions is completely received from one of the connected remote sites; Another optional embodiment comprises directing the test data to one of any number of designated expert interpreters, each of whom would be prompted to read the test results assigned to them.

### Preparation of the Genetic Testing Data for Transmission

After gathering and verifying the genetic testing data, the data collection system 300 prepares the genetic testing data for transmission to the central data analysis/interpretation system 600 for interpretations and reports. Depending on 1) preferences or capabilities of the remote location, 2) the type of genetic testing data to be transmitted, and 3) the transmission system to be used, the preparation of the genetic testing data by the data collection system 300 for transmission could take alternative forms.

One alternative is for the data collection system 300 to mask all patient-identifying information that could be used to identify the patient, which is contrary to desired or mandated privacy requirements, from other data related to the genetic test of the patient. Under this alternative, information transmitted to the central data analysis/interpretation system 600 does not include any information that could identify the patient. When interpretations and reports (described below) are returned from central data analysis/interpretation system 600, the data collection system 300 at the remote location could then correlate the data identifying a patient data with the test result at the remote site. In this manner, no genetic testing or demographic data positively linked to a named patient ever leaves the local clinical laboratory. This arrangement greatly increases the private nature of the entire remote genetic testing procedure.

Another alternative is for the data collection system 300 to separate the data into two separate files that can be transmitted separated. One of the files can include information on the patient that is not generally viewed as confidential, such as the patient's name, address, job, age sex, weight and third party payer information, while the other file can include patient data that is confidential such as genetic testing data and medical history. The file containing the confidential information does not include any information that identifies the patient. After the data is separated, the two files of information can be transmitted separately, including the transmission of information through different transmission modalities and at different times. Upon receipt of the two files, the central data analysis/interpretation system 600 can then correlate the two files to perform the required interpretations and analysis necessary to generate the reports (described below). Under this alternative, the central data analysis/interpretation system 600 has all of the pertinent information on the patient, yet no genetic testing or other confidential data on a patient that could identify the patient is ever transmitted.

Another alternative is for the data collection system 300 to encrypt, using any convenient encryption technology, any portion of the information to be transmitted. The encrypted transmission is deciphered by the analysis/interpretation system 600 upon receipt.

Any or all of these data preparation alternatives may be used in any desired combination to ensure the safe, secure and confidential transmission of the genetic testing data and other information from the remote location to the analysis/interpretation system 600.

### Transmission of the Genetic Testing Data

The raw and non-interpreted, and possibly encrypted, data is then transmitted, either automatically or initiated by an operator, to a central data analysis/interpretation system 600, which may be in a location different from the clinical laboratory that performs the testing processes described above. This transmission may be accomplished using any convenient data transmission scheme. In a preferred embodiment, a remote, secure Internet portal 400 is provided and accessed from the clinic location. In another preferred embodiment, conventional application service provider architecture is used by the central data analysis/interpretation system 600 to service an application 400 running on the clinic data collection system 300.

Regardless of the data transmission scheme chosen, the functions of this component of the system include the distinct capture of the raw and non-interpreted analytic data and the pertinent patient demographic data. The transmission, processing interpretation and report of these data, including the maintenance of each type of data in a secured and confidential form is an important embodiment of the invention. FIG. 12 shows one embodiment of the invention, in which genetic testing information is transmitted over the Internet as a non-limiting example of a computer network.

### Data Interpretation

Medical and genetic experts resident at a central location read and interpret the genetic data transmitted from the remote locations and determine the genetic profile of the patient. In addition, the demographic information on the patient provided with the genetic data greatly enhances the ability of the medical and genetic experts resident at a central location to provide additional useful advice in the reports to clinical professional (physician) and the patient as discussed below.

Once the data has been interpreted, a series of reports 700 are generated and securely transmitted back to the source clinic. While any secondary data transmission scheme may be used, the preferred approach is to use the same data transmission scheme as used to transmit the data from the source clinical laboratory to the central data interpretation facility, i.e., a two-way communications scheme.

Typical contents of the reports include the analytic measurements of the genetic tests themselves; insurance reimbursement data (e.g., recommended CPT coding for the procedures that have been performed); and genetic counseling. In a preferred embodiment, both a technical report directed to the clinical professional (physician), and a separate non-technical report directed to the patient, are included. The system may identify technical problems in the performance of the test steps done at the remote location that require added samples or steps to ensure a quality test result. Such problems may then be identified to a remote location so that corrective actions may be taken.

Another aspect of the invention is the manner is which additional information derived from the medical and scientific literature is incorporated into the construction of customized interpretations and comments. The expert interpreter of these tests relies in whole or in part on an integral expert system database, which contains large amounts of prewritten information pertaining to various clinical and pathologic aspects of the condition being tested. The expert system is part of the invention and is described in detail below.

The expert system is integral to the genetic testing system. In general terms, the expert system is an electronic database constructed from one or more of a variety of commercially available software products. The database is derived from the manual and automated review of the medical and scientific literature made available from the variety of the public and subscription based information search sources. The database collates information from the various sources based on prescribed keywords that a are specific the disease, test and clinical condition. The database sort is modified according to the results obtained from a particular patients test, and in combination with the provided demographic information. Hence the expert system contains information in excess of what is needed for any one patient sample, and uses the specific results, transmitted from the remote lab to initiate a sort of pertinent references and comments that in turn are presented to the expert interpreter.

The expert system integrates with other aspects of the system at the points involving the sorting of patient demographic and analytic data. The interface involves the initiation of a primary sort of pertinent comments based on the specific analytic result from one patient. The primary sorting derives, from a large data set, a subset of information such as risk and therapeutic options that is based on the specific gene test results. When provided, the demographic data, including information such as gender, age, medications and pre-existing medical conditions, initiates subsequent sorts of the database. The subsequent sorts further reduce the set of selected comments and references to those pertinent to all of the provided demographic and analytic data conditions. The result is a markedly reduced set of prewritten interpretations and comments that the expert can then use in the creation of the customized genetic test report.

The expert system 650, which is a subsystem to the complete system, contains entries from the medical literature derived from public and commercial sources (such as Medline, PubMed, Compendex, GeneBank, www.genetest.com and www.webmd.com). From searches performed within these sources, the expert system abstracts selected information about a particular disease or genetic condition. The expert system involves the assignment of various categories of the derived data that are used in the sort function. Such categories include, but are not limited to disease association with a particular genetic result, risk calculations about diseases given a certain demographic or analytic test result, and options for therapy.

We claim:

1. A method of performing genetic testing in a telemedicine model, in which test results are transmitted to a central location at which expertise in interpretation of genetic testing resides.

2. A method of genetic testing in which genetic source material is tested at a location different from a location at which analysis of results of the testing and preparation of interpretive genetic testing reports occurs.

3. A genetic test data gathering process that, automatically or as initiated by a remote site or central location, gathers from a remote site database genetic data relevant to a desired genetic test to be performed at the remote site and subsequently interpreted at the central location.

4. An interpretive report generator:
(a) a database 823, accessible by an expert system 650, the database containing genetic reference information 818;
(b) a clinical data collection system 300 containing patient data 407, the clinical data collection system accessible to the database for storage of the patient data in the database;
(c) the expert system comprising a control mechanism for (i) inspecting the patient data in the database, (ii) searching the genetic reference information stored in the database, said search parameters based on the patient data, and (iii) generating a data report 700, which contains an interpretation of the patient data, the data report accessible to the clinical data collection system.

5. The interpretive report generator of claim 4, wherein the generated data report is analyzed by a health care professional and revised as may be deemed necessary by the health care professional.

6. The method of performing genetic testing in a telemedicine model of claim 1, wherein the genetic test data is generated according to a protocol of an integrated test kit in conjunction with other components of the kit.

7. The method of performing genetic testing in a telemedicine model of claim 6, also comprising a method of (a) entry of patient demographic data, (b) ordering a genetic test, (c) ordering kit components, (c) calculating reagent volumes as a function of the number of test specimens, (d) calculating reagent reaction times, (e) presentation of protocol steps, (f) reading the genetic test data presented by denatured and marked nucleic acid, (g) analyzing the nucleic acid test data, (h) analyzing sorting and selecting, according to an expert system algorithm, information from a genetic database to interpret the nucleic acid test data and generate an initial interpretive report, and (i) generating a final interpretive report of a licensed physician.

8. The method of claim 6, wherein bar code labeled components of the integrated test kit can be ordered from a remote site electronically.

9. A test kit for performing a molecular genetic assay of a specimen, comprising components for (i) purification of specimen nucleic acid and (ii) denaturing and marking the purified nucleic acid, so that characteristics of target genes can be identified.

10. The components of the test kit of claim 9, comprising an extraction rack, a mixing rack, a microtitor, and an assay protocol.

11. The test kit of claim 10, also comprising components from the group including a specimen sample, liquid collection media, treated paper for cell capture, template guide, graduated transfer pipette, microcentrifuge tube, a kit box, filter basket, mineral oil, wax; label, genetic test order form, component order form, pipette tip, tube snap top, assay protocol, pipette tip, skirted microtitor base, extractor rack face plate, mixing rack face plate, a record and store device, training material, calculator tool, disposable materials, reagents such as protein buffer, probe, oligonucleotide, first fluorescent marker, second fluorescent marker, and thermostable endonucleolytic enzyme, and containers for certain of the foregoing components in the group.

12. The test kit of claim 10, wherein at least one of the components is bar coded with information relating to the component.

13. The test kit of claim 9, also comprising a device to record and store information about the kit selected from the group consisting of material safety, quality control, expiration, environmental condition as a function of time and duration, and location as a function of time and duration.

14. An extractor rack for purification of nucleic acid, comprising a rack body constructed of a material and a face of the rack body comprised of a matrix of adjacent rows and adjacent columns with sized and spaced apertures at each intersection of a row and column.

15. The extractor rack of claim 14, wherein the number of rows corresponds to the number of specimens from which nucleic acid is to be extracted.

16. The extractor rack of claim 15, wherein each specimen is confined to one row.

17. The extractor rack of claim 15, wherein there is a column for the blood specimen.

18. The extractor rack of claim 17, wherein there is a column for each successive steps in the process of extraction of nucleic acid from a specimen.

19. The extractor rack of claim 18, wherein a first column contains blood specimens, a second column adjacent to the first column contains a buffy coat fraction, a third column adjacent to the second column contains a wash solution and a blood fraction, a fourth column adjacent to the third column contains a wash and an elution solution and a blood fraction; and a fifth column adjacent to the fourth column contains an elution solution and the purified nucleic acid.

20. The extractor rack of claim 14, wherein each column is labeled to indicate its contents or its function.

21. The extractor rack of claim 14, including at least one aperture, in addition to those at the intersections of the rows and columns, for containment of at least one reagent as may be required for a specific genetic test, such as red blood cell lysis, nucleated cell lysis buffers, washing buffer, nucleic acid elution solution, or hydration buffers.

22. The extractor rack of claim 14, wherein the first step in the purification of the nucleic acid of a specimen in a row is performed in a column adjacent to the column in which the specimen is located and each successive step in the purification process is performed in a column adjacent to the column where the previous step was performed.

23. The extractor rack of claim 14, wherein a plurality of specimens, each in a separate row, is purified by (i) moving a first specimen in a first row of a first column to an adjacent second column in the first row where the first specimen is processed according to a first protocol step, moving a second specimen in a second row of the first column to the adjacent second column in the first row where the specimen is processed according to the first protocol step, and moving each remaining specimen in successive rows to the adjacent second column in each respective row where each specimen is processed according to the first protocol step and (ii) moving each specimen in the manner described in subsection (i) of this claim to each of the columns in the extractor rack where each specimen is processed in each column according to a protocol step specified for each column.

24. The extractor rack of claim 14, wherein the number of rows is eight.

25. The extractor rack of claim 14, wherein the spacing between the columns, rows, and apertures within each column and row is the same as the spacing between the columns, rows, and apertures within each column and row of a microtiter into which specimen fractions are to be transferred.

26. The extractor rack of claim 16, wherein the apertures for containing the specimen are sized to accommodate the specimen container.

27. The extractor rack of claim 18, wherein the apertures in the columns for the successive steps in the process of extraction of nucleic acid from a specimen are sized to accommodate a microcentrifuge tube.

28. The extractor rack of claim 17, wherein there is a column for each successive steps in the process of extraction of nucleic acid from a specimen.

29. The extractor rack of claim 14, wherein the rack body is dimensioned to be of a depth to accommodate the length of the tubes in the apertures.

30. The extractor rack of claim 14, wherein the apertures are spaced to accommodate a multichannel pipette without realignment of the multichannel pipette tips for transfer of aliquoted amounts of specimen fractions from column to column and from the elution and DNA column to a microtitor.

31. A mixing rack for creation of a master mix and for organizing control samples, comprising a rack body constructed of a material and a matrix of sized and spaced apertures in a face of the rack body.

32. The mixing rack of claim 31, also comprising a face plate.

33. The mixing rack of claim 32, wherein the face plate is releasably adhered to the face of the rack body in which the matrix of sized and spaced apertures is located.

34. The mixing rack of claim 32, wherein the number of apertures in the face plate is equal to or less than the number of apertures in the matrix of sized and spaced apertures in the rack body face.

35. The mixing rack of claim 34, wherein a plurality of apertures in the face plate are in registry with the apertures in the rack body face.

36. The mixing rack of claim 31, wherein the apertures are sized for containing microcentrifuge tubes.

37. The mixing rack of claim 31, wherein the rack is insulated.

38. The mixing rack of claim 31, configured for processing one specimen at a time.

39. The mixing rack of claim 33, wherein the face plate is labeled to guide sequencing of protocol steps for the creation of a master mix and for organizing control specimens, whereby the master mix and the control specimens may be used to mark and denature nucleic acid for Factor V Leiden and Factor II genetic tests.

40. The mixing rack of claim 38, wherein the face plate is comprised of (i) a first column labeled, Factor V Leiden controls, of four apertures, the first aperture labeled, wild type, the second aperture labeled, HET (heterozygot), the third aperture labeled, mutant type, and the fourth aperture labeled, no target; (ii) a second column labeled, Factor II controls, of four apertures adjacent the first column, each aperture labeled the same as the corresponding aperture in the first column; (iii) a third column of seven apertures, the first aperture labeled, buffer, the second labeled, probe, the third labeled, oligo, the fourth labeled, Fret 1, the fifth labeled, Fret 2, the sixth labeled, cleavage enzyme, and the seventh labeled, Factor V Leiden master mix; and (iii) a fourth column of seven apertures adjacent the third column, each of the first six apertures labeled the same as the corresponding apertures in the third column and the last aperture labeled, Factor II master mix.

41. The mixing rack of claim 40, wherein the first column labeled, Factor V Leiden controls, is the same color as the area around the aperture labeled, Factor V Leiden master mix, in the third column and the second column labeled, Factor II controls, is the same color as the area around the aperture labeled, Factor II master mix, in the fourth column.

42. The mixing rack of claim 41, wherein the area around each successive aperture in the first and third columns is a successively darker shade of the color of the first and third columns and the area around each successive aperture in the second and fourth columns is a successively darker shade of the color of the second and fourth columns.

43. The process of creating a master mix comprising sequentially transferring, according to steps of a protocol, to a master mix container in a mixing rack column an aliquoted amount of a (i) protein buffer, (ii) probe, (iii) oligonucleotide, (iv) first fluorescent marker, (v) second fluorescent marker, and (vi) thermostable endonucleolytic enzyme from each container of the foregoing reagents i through vi, which containers are located in the same column as the master mix container.

44. The mixing rack of claim 31, wherein the spacing between apertures within each column of the mixing rack is the same as the spacing between apertures in a microtiter, whereby control samples contained in the mixing rack may be efficiently transferred to the microtiter.

45. The mixing rack of claim 31, wherein the rack body is dimensioned to be of a depth to accommodate the length of the tubes in the apertures.

46. A microtitor for containment of specimen purified nucleic acid, master mix, and specimen controls.

47. The microtitor claim 46, also comprising a skirted base

48. A template guide comprising a material; a matrix of rows and columns of apertures, the apertures spaced to correspond to the center-to-center distance between the microtiter wells; and labeling.

49. The template guide of claim 48, wherein the guide has a template on each side.

50. The template guide of claim 48, also comprising reference marks to guide alignment of the template with the microtiter.

51. The template guide of claim 48, labeled to guide a reaction in the presence of mutant nucleotides.

52. The template guide of claim 48, labeled to guide a reaction in the presence of wild type nucleotides.

53. The template guide of claim 48, labeled to guide a genetic reaction without the presence of an enzyme.

54. The template guide of claim 48, labeled to guide a genetic reaction in the presence of an enzyme.

55. The template guide of claim 48, wherein the apertures are numbered.

56. The template guide of claim 48, labeled to guide a reaction for a plurality of purified specimens.

57. A kit box means for packaging, shipping, and storage of components; organizing the components; and using the components to perform genetic tests.

58. A kit box comprising compartments enclosed within the box and a cover, so that the kit components are contained in the compartments in an organized manner.

59. The kit box of claim 58 also comprising a means for removably supporting a protocol manual.

60. The kit box of claim 58 wherein the support means is affixed on the inside of the cover.

61. The kit box of claim 58 wherein the support means is a bench stand with a triangular base to stabilize the stand on the bench, hold open the pages of the protocol manual, allow the pages to be turned, and to maintain the desired pages open.

62. The kit box of claim 58, wherein one or more of the compartments are insulated.

63. The kit box of claim 58, configured for testing batches of specimens of various batch sizes.

64. An assay protocol, comprising steps for operating a genetic test kit to perform genetic tests.

65. The protocol of claim 64, wherein the protocol is recorded in electronic media.

66. The protocol of claim 65, wherein the protocol is implemented in a software application.

67. The protocol of claim 64, wherein the protocol is recorded in hard copy media.

68. The protocol of claim 64, wherein at least one-step is described in detail in text and illustration.

69. The protocol of claim 68, wherein the text and the illustration are side-by-side.

70. The protocol of claim 64, also comprising a guide bar.

71. The protocol of claim 70, wherein a guide bar is visually proximate the detailed description of each step.

72. The guide bar of claim 70, comprising a short text statement of at least one step of the protocol.

73. The guide bar of claim 72, comprising a short text statement for each of the steps of the protocol.

74. The guide bar of claim 70, also comprising a slide bar.

75. The guide bar of claim 70, comprising a short text statement for each of the steps of the protocol with the step described in detail marked on the slide bar.

76. The guide bar of claim 75, wherein the short text statement of the step described in detail is marked on the slide bar by an illustration of a magnifying glass.

77. The protocol of claim 64, also comprising forms for (i) entry of patient demographic data, (ii) presentation of genetic test data, and (iii) interpretation of genetic test data.

78. The protocol of claim 64, also comprising a tool for calculation of (i) the volumes of reagents used for purification, marking, and denaturing nucleic acids upon entering the number of specimens in a test batch and (ii) reaction times.

79. The protocol of claim 64, also comprising a form for ordering genetic tests and genetic test kit components comprising questions to be answered by the ordering person, said questions designed to elicit patient demographic data and other information necessary for a licensed physician to determine whether a genetic test is indicated and if so the appropriate test and kit components.

80. A form for ordering genetic tests and genetic test kit components comprising questions to be answered by the ordering person, said questions designed to elicit patient demographic data and other information necessary for a licensed physician to determine whether a genetic test is indicated and if so the appropriate test and kit components.

81. The ordering form of claim 80, wherein the form is in an electronic media and its contents are sent electronically to a remote site.

82. The ordering form of claim 1, wherein the contents of the ordering form are inputed into an expert system having a database of genetic information for initial evaluation of whether a genetic test is indicated and if so the appropriate test and kit components and subsequent licensed physician review and approval, disapproval, or revision of the initial evaluation.

83. A tool for calculation of (i) the volumes of reagents used for purification, marking, and denaturing nucleic acids upon entering the number of specimens in a test batch and (ii) reaction times.

84. The tool of claim 83, wherein the tool is a software application

85. The tool of claim 84, wherein the tool is a software application accessible at an Internet site.

86. A method of purifying nucleic acid, comprising performing a series of steps according to a laboratory assay protocol in conjunction with using an extractor rack.

87. A method of purifying the nucleic acid of a whole blood specimen, comprising the steps of (a) placing a container of at least one specimen in a first row in a specimen column of an extractor rack, (b) transferring an aliquot amount of the specimen to a first row container in a buffy coat column of the extractor rack, centrifuging the buffy coat container to fractionate the specimen into plasma, buffy coat, and red blood cells, returning the centrifuged container to the first row in the buffy coat column, and transferring an aliquot amount of the buffy coat into a filter basket in a container in the first row in a wash column of the extractor rack, (b) adding wash solution to the center of the filter basket, centrifuging the wash container until a red tinged fluid collects at the bottom of the container, and transferring the filter basket to a container in its respective row in a wash and elution column of the extractor rack, (c) adding a wash solution to the wash and elution container, centrifuging the container, adding elution solution, centrifuging the wash and elution container again, and transferring the filter basket from the wash and elution container to a container in its respective row in an elution and DNA/RNA column of the extractor rack, and (d) adding elution solution, heating the elution and DNA/RNA container, centrifuging the container, returning the container to its respective row in the elution and DNA/RNA column, and discarding the filter basket.

88. A method of denaturing and marking purified nucleic acid, comprising performing a series of steps according to a laboratory assay protocol in conjunction with using a mixing rack and a microtitor.

89. A method of denaturing and marking purified nucleic acid for the Factor V Leiden genetic assay, comprising the steps of (a) transferring a mixed, aliquoted amount of each of the unfrozen protein buffer, probe, olgionucleotide, first FRET, second FRET, and cleavage enzyme reagents and each of the wildtype, HET, mutant, and targetless control samples into containers in the respectively labeled aperture in a mixing rack, centrifuging each of the containers, and returning the containers to their respective aperture, (b) transferring an aliquoted amount of each of the reagents, one at a time, to the container in the master mix aperture in the mixing rack, mixing the master mix contents, centrifuging the master mix, and returning the centrifuged master mix container to its respective aperture. (d) transferring an aliquoted amount of wild type control sample to a microtitor well in a first column, HET control sample to a second well in the same column, mutant control sample to a third well in the same column, and targetless control sample to a fourth well in the same column, (e) transferring an aliquoted amount of purified nucleic acid from the elution and DNA column of the extractor rack to four wells in a second column adjacent to the first column in the microtitor, each of the four wells in a row adjacent the wells of the first column, (f) dispensing an aliquoted amount of mineral oil on top of each of the control sample wells and each of the purified nucleic acid wells, (g) heating the microtitor for a first period of time at a first temperature, (h) while heating the microtitor at a second lower temperature, transferring an aliquoted amount of master mix to each of the control samples and each of the purified nucleic acid wells by extending the tip of a pipette containing the master mix below the mineral oil, (i) continuing to heat the microtitor at the second temperature for a second period of time to incubate.

90. A method of reading the incubated microtitor wells of claim, comprising placing the microtitor in a fluorometer and initiating reading.
